(19) United States
(12) Patent Application Publication
(10) Pub. No.: US 2004/0002502 A1
(43) Pub. Date: Jan. 1, 2004

(54) Medicament combinations comprising heterocyclic compounds and a novel anticholinergic

(75) Inventors: Rolf Banholzer, Stuttgart (DE); Helmut Meissner, Ingelheim (DE); Gerd Morschhaeuser, Biberach (DE); Michael P. Pieper, Biberach (DE); Gerald Pohl, Biberach (DE); Richard Reichl, Gau-Algesheim (DE); Georg Speck, Ingelheim am Rhein (DE); Christopher John Montague Meade, Bingen (DE); Michel Pairet, Biberach (DE)

Correspondence Address: BOEHRINGER INGELHEIM CORPORATION, 900 RIDGEBURY ROAD, P. O. BOX 368, RIDGEFIELD, CT, 06877, US

(73) Assignee: Boehringer Ingelheim Pharma GmbH & Co. KG, Ingelheim (DE)

(21) Appl. No.: 10/409,402

(22) Filed: Apr. 8, 2003

(30) Foreign Application Priority Data
Apr. 12, 2002 (DE) 102 16 427

(51) Int. Cl. A61K 31/506; A61K 31/496; A61K 31/4433; A61K 31/4745; A61K 31/4709; A61K 31/343; A61K 31/353

(52) U.S. Cl. 514/254.11; 514/291; 514/275; 514/307; 514/337; 514/456; 514/469

(57) ABSTRACT

The present invention relates to novel pharmaceutical compositions based on a new anticholinergic 1 and heterocyclic compounds 2, processes for preparing them and their use in the treatment of respiratory complaints.

### BACKGROUND OF THE INVENTION

The present invention relates to novel pharmaceutical compositions based on a new anticholinergic 1 and heterocyclic compounds 2, processes for preparing them and their use in the treatment of respiratory complaints.

### BRIEF SUMMARY OF THE INVENTION

The present invention relates to novel pharmaceutical compositions based on a new anticholinergic of formula 1

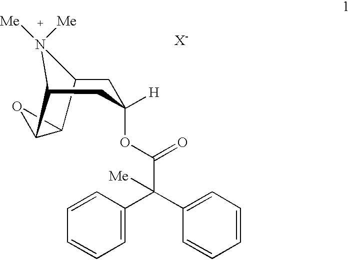

wherein X⁻ may have the meanings given hereinafter and the heterocyclic compounds of formula 2

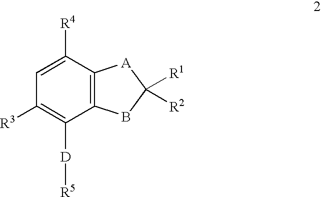

wherein the groups A, B, D, R¹, R², R³, R⁴ and R⁵ may have the meanings given in the claims and specification, processes for preparing them and their use in the treatment of respiratory complaints. The compounds of formula 2 are known from WO 96/36624.

Within the scope of the present invention the salts of formula 1

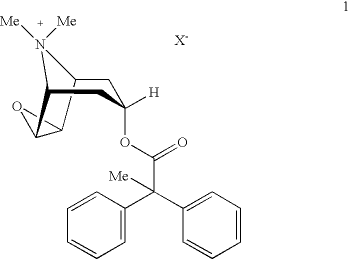

wherein

X⁻ denotes an anion with a single negative charge, preferably an anion selected from the group consisting of chloride, bromide, iodide, sulphate, phosphate, methanesulphonate, nitrate, maleate, acetate, citrate, fumarate, tartrate, oxalate, succinate, benzoate and p-toluenesulphonate

are used as the anticholinergic.

Preferably, the salts of formula 1 are used wherein

X⁻ denotes an anion with a single negative charge selected from among the chloride, bromide, 4-toluenesulphonate and methanesulphonate, preferably bromide.

Most preferably, the salts of formula 1 are used wherein

X⁻ denotes an anion with a single negative charge selected from among the chloride, bromide and methanesulphonate, preferably bromide.

Particularly preferred according to the invention is the salt of formula 1 wherein X⁻ denotes bromide.

### BRIEF DESCRIPTION OF THE DRAWINGS

FIG. 1 is an exploded view of an inhaler for use with a medicament in powder form for inhalation.

FIG. 2a shows a longitudinal section of a nebuliser for administration of a medicament in liquid form, with its spring biased.

FIG. 2b shows a longitudinal section of a nebuliser for administration of a medicament in liquid form, with its spring relaxed.

### DETAILED DESCRIPTION OF THE INVENTION

Anticholinergics may appropriately be used to treat a number of diseases. Particular mention should be made, for example, of the treatment of asthma or COPD (chronic obstructive pulmonary disease). For treating these diseases WO 92/16528 proposes, for example, anticholinergics which have a scopine, tropenol or tropine basic structure.

The problem on which WO 92/16528 is based is the preparation of anticholinergically active compounds which are characterised by their long-lasting activity. To solve this problem WO 92/16528 discloses inter alia benzilic acid esters of scopine, tropenol or tropine.

For treating chronic diseases it is often desirable to prepare pharmaceutical compositions with a longer-lasting effect. This will generally ensure that the concentration of the active substance needed to achieve the therapeutic effect is present in the body for a longer period of time without the need for the pharmaceutical composition to be administered repeatedly and all too frequently. Moreover, if an active substance is administered at longer intervals of time, this contributes to the feeling of well-being of the patient to a considerable degree. It is particularly desirable to provide a pharmaceutical composition which can be used to therapeutically good effect by administering it once a day (single dose). A single application per day has the advantage that the patient can become accustomed relatively quickly to the regular taking of the medicament at a particular time of the day.

If it is to be used as a medicament for administration once a day, the active substance which is to be given must meet particular requirements. First of all, the desired onset of the activity after the administration of the pharmaceutical composition should occur relatively quickly and ideally the activity should remain as constant as possible over a fairly lengthy ensuing period. On the other hand the duration of activity of the pharmaceutical composition should not greatly exceed a period of about one day. Ideally, an active substance should have an activity profile such that the preparation of a pharmaceutical composition which is intended to be administered once a day and contains the active substance in therapeutically appropriate doses can be properly controlled.

It has been found that the esters of scopine, tropenol or tropine disclosed in WO 92/16528 do not meet these more stringent requirements. Because of their extremely long duration of activity, significantly exceeding the period of about one day specified above, they cannot be used therapeutically in a single once-a-day dose.

Surprisingly, an unexpectedly beneficial therapeutic effect, particularly a synergistic effect can be observed in the treatment of inflammatory and/or obstructive diseases of the respiratory tract if the anticholinergic of formula 1 is used with one or more, preferably one, compound of formula 2. In view of this synergistic effect the pharmaceutical combinations according to the invention can be used in smaller doses than would be the case with the individual compounds used in monotherapy in the usual way.

The effects mentioned above may be observed both when the two active substances are administered simultaneously in a single active substance formulation and when they are administered successively in separate formulations. According to the invention, it is preferable to administer the two active substance ingredients simultaneously in a single formulation. The pharmaceutical compositions according to the invention are preferably administered by inhalation.

Within the scope of the present invention, any reference to the compound 1′ is to be regarded as a reference to the pharmacologically active cation of the following formula contained in the salts 1:

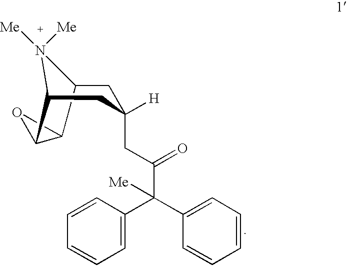

Any reference to compounds 1 naturally also includes a reference to the cation 1′.

In the pharmaceutical combinations mentioned above the active substances 1 and 2 may either be combined in a single preparation or contained in two separate formulations. Pharmaceutical compositions which contain the active substances 1 and 2 in a single preparation are preferred according to the invention.

Accordingly, in one aspect, the present invention relates to a pharmaceutical composition, characterised in that it contains an anticholinergic of formula 1 in combination with one or more, preferably one, compound of general formula 2

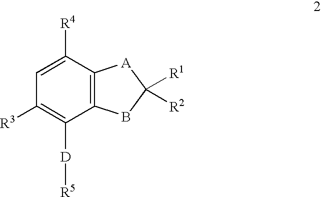

wherein

R¹ denotes hydrogen, methyl, ethyl, n-butyl, i-butyl, phenyl, 2-ethylphenyl, 2-i-propylphenyl, benzyl, 4-pyridyl, 2-pyridyl, —CO-phenyl, CN, or

together with R² denotes a butylene or pentylene bridge;

R² denotes hydrogen, methyl, ethyl, or

together with R¹ denotes a butylene or pentylene bridge, or

together with R¹³ denotes a single bond or a butylene bridge;

R³ denotes hydrogen;

R⁴ denotes methoxy;

R⁵ denotes cyclohexyl, phenyl, 3-methoxycarbonylphenyl, 4-methoxycarbonylphenyl, 3-carboxyphenyl, 4-carboxyphenyl, CN, —COOH, —COOMe, —COOEt, 3,5-dichloro-pyridin-4-yl, 4-pyridyl or 4-pyridyl-N-oxide;

A denotes oxygen or —CH₂—;

B denotes oxygen or one of the groups —C(R¹²)(R¹³) or —CH(R¹⁵)—CH(R¹⁷);

D denotes a group selected from —CH₂—CH₂—, —CH(Ph)—CH₂—, —CONH—, —CO—CH₂—, —CH═CH—, —C(Ph)═CH—, —C(CR¹⁸)(CR¹⁹)—X—, —C(R^{19a})═Y—, C═C— or phenylene;

R¹² denotes hydrogen, methyl, ethyl, i-propyl, phenyl or —CH₂—COR^{x};

R¹³ denotes hydrogen or

together with R² denotes a single bond or a butylene bridge

R¹⁵ denotes hydrogen or

together with R¹⁷ denotes a single bond;

R¹⁷ denotes hydrogen or

together with R¹⁵ denotes a single bond;

R¹⁸ denotes hydrogen or methyl;

R¹⁹ denotes hydrogen, methoxy, phenyl or CN;

R^{19a} denotes hydrogen, methyl or phenyl;

R^{x} denotes hydroxy, ethoxy, benzyloxy, 2-phenylethyloxy, 4-methylpiperazin-1-yl, 4-phenylpiperazin-1-yl, N-tetrahydroisoquinolinyl, —NH-phenyl, —NH—benzyl, —NH—CH₂-(4-methoxyphenyl), —NH—CH₂-(4-fluorophenyl), —NH—CH₂-(4-chlorophenyl), —NH—CH₂-(2-chlorophenyl), —NH-(3-pyridyl), —NH—CH₂-(2-pyridyl), —NH—CH₂-(3-pyridyl), —NH—CH₂-(4-pyridyl), —NH-(3,5-dichloropyridin-4-yl) or —NH-(2-pyrimidinyl);

X denotes —CH₂—, —S— or —NH—

Y denotes CH, CCN, CCOOEt or CHCONH,

optionally in the form of the individual optical isomers, mixtures thereof or racemates and optionally in the form of the pharmacologically acceptable acid addition salts thereof.

Compounds of general formula 2 wherein R³ denotes hydrogen and R⁴ denotes methoxy and wherein A represents oxygen, B the group —C(R¹²)(R¹³)— and D the group —CONH—, have the general formula 2a

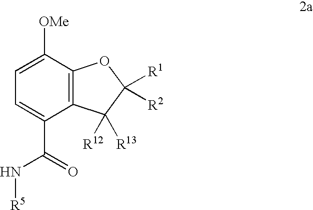

In a preferred aspect the present invention relates to a pharmaceutical composition, characterised in that it contains an anticholinergic of formula 1 in combination with one or more, preferably one, compound of general formula 2a

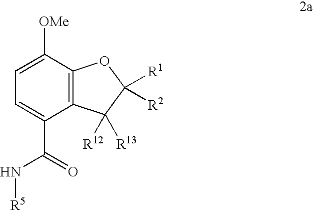

wherein

R¹ denotes hydrogen, n-butyl, benzyl, 4-pyridyl, 2-pyridyl, —CO-phenyl or CN;

R² denotes hydrogen or together with R¹³ denotes a single bond;

R⁵ denotes cyclohexyl, phenyl, 3,5-dichloro-pyridin-4-yl or 4-pyridyl;

R¹² denotes hydrogen, methyl, ethyl, i-propyl, phenyl or —CH₂—COR^{x};

R¹³ denotes hydrogen or together with R² denotes a single bond;

R^{x} denotes hydroxy, ethoxy, benzyloxy, 2-phenylethoxy, 4-methylpiperazin-1-yl, 4-phenylpiperazin-1-yl, N-tetrahydroisoquinolinyl, —NH-phenyl, —NH-benzyl, —N H—CH₂-(4-methoxyphenyl), —N H—CH₂-(4-fluorophenyl), —N H—CH₂-(4-chlorophenyl), —NH—CH₂-(2-chlorophenyl), —NH-(3-pyridyl), —NH—CH₂-(2-pyridyl), —NH—CH₂-(3-pyridyl), —NH—CH₂-(4-pyridyl), —NH-(3,5-dichloropyridin-4-yl) or —NH-(2-pyrimidinyl),

optionally in the form of the individual optical isomers, mixtures thereof or racemates and optionally in the form of the pharmacologically acceptable acid addition salts thereof.

Particularly preferably, the present invention relates to a pharmaceutical composition which contains, in addition to an anticholinergic of formula 1, one or more, preferably one, compound of general formula 2a which is selected from the compounds according to Table 1.

TABLE 1
Particularly preferred compounds of formula 2a

| Example | R¹ | R² | R¹³ | R¹² | R⁵ |
|---|---|---|---|---|---|
| 1 | —H | —H | —H | —H | 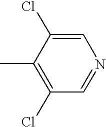 |
| 2 | —H | —H | —H | —Me | 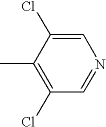 |
| 3 | —H | —H | —H | —Et | 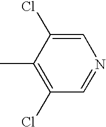 |
| 4 | —H | —H | —H | -iso-Pr | 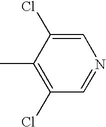 |
| 5 | —H | —H | —H | —CH₂CO₂—Et | 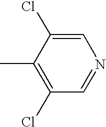 |
| 6 | —H | —H | —H | —CH₂CO₂—Et | 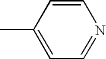 |
| 7 | —H | —H | —H | —CH₂CO₂—Et | phenyl |
| 8 | —H | —H | —H | —CH₂CO₂—Et | 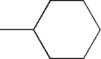 |
| 9 | —H | —H | —H | —CH₂CO₂H | 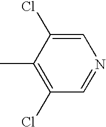 |
| 10 | —H | —H | —H | —CH₂CO₂H | 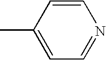 |
| 11 | —H | —H | —H | —CH₂CO₂H | phenyl |
| 12 | —H | —H | —H | —CH₂CO₂H | 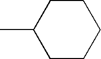 |
| 13 | —H | —H | —H | —CH₂CO₂benzyl | 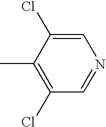 |
| 14 | —H | —H | —H | —CH₂CO₂benzyl | 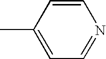 |
| 15 | —H | —H | —H | —CH₂CO₂benzyl | phenyl |
| 16 | —H | —H | —H | —CH₂CO₂benzyl | 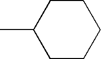 |
| 17 | —H | —H | —H | 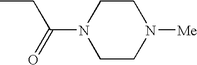 | 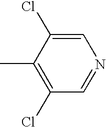 |
| 18 | —H | —H | —H | 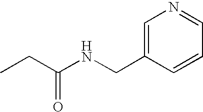 | 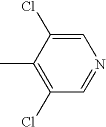 |
| 19 | —H | —H | —H | 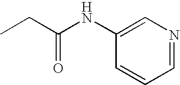 | 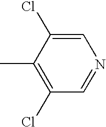 |
| 20 | —H | —H | —H | 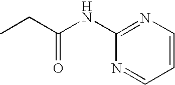 | 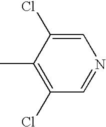 |
| 21 | —H | —H | —H | 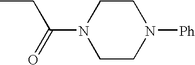 | 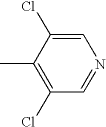 |
| 22 | —H | —H | —H | 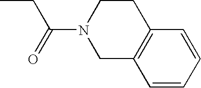 | 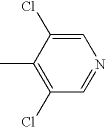 |
| 23 | —H | —H | —H | 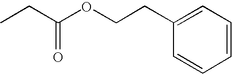 | 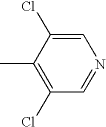 |
| 24 | —H | —H | —H | 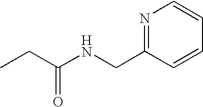 | 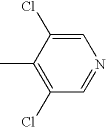 |
| 25 | —H | —H | —H | 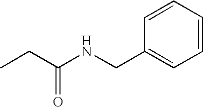 | 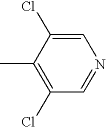 |
| 26 | —H | —H | —H | 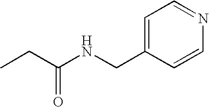 | 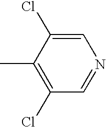 |
| 27 | —H | —H | —H | 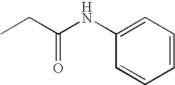 | 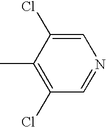 |
| 28 | —H | —H | —H | 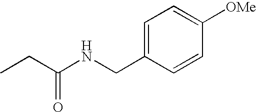 | 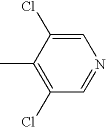 |
| 29 | —H | —H | —H | 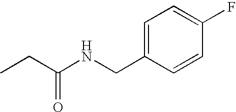 | 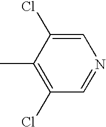 |
| 30 | —H | —H | —H | 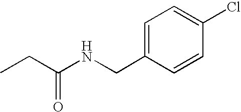 | 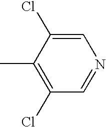 |
| 31 | —H | —H | —H | 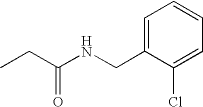 | 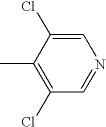 |
| 32 | —H | —H | —H | 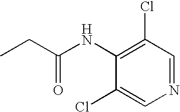 | 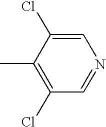 |

| | | | | |
|---|---|---|---|---|
| 33 | —H | single bond | —H | 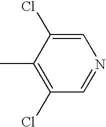 |
| 34 | —CN | single bond | —H | 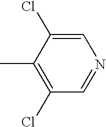 |
| 35 | —CO—phenyl | single bond | —H | 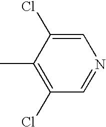 |
| 36 | -n-butyl | single bond | —H | 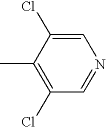 |
| 37 | —benzyl | single bond | —H | 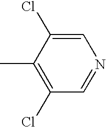 |
| 38 | 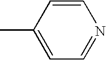 | single bond | —H | 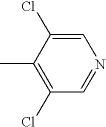 |
| 39 | 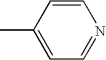 | single bond | —H | 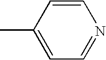 |
| 40 | 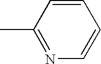 | single bond | —H | 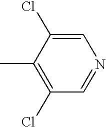 |
| 41 | 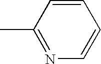 | single bond | —H | 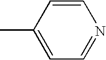 |
| 42 | —H | single bond | —phenyl | 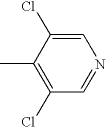 |
| 43 | —H | single bond | —CH₂CO₂—Et | 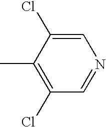 |
| 44 | -H | single bond | —CH₂CO₂H | 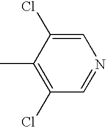 |

Compounds of general formula 2 wherein R³ denotes hydrogen and R⁴ denotes methoxy and wherein A denotes oxygen, B denotes the group —C(R¹²)(R¹³)— and D denotes the group —C(R¹⁸)(R¹⁹)—X—, have the general formula 2b

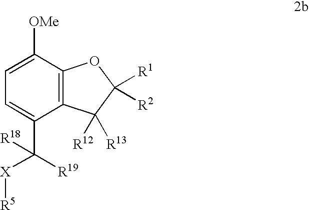

In a preferred aspect the present invention the present invention relates to a pharmaceutical composition, characterised in that it contains an anticholinergic of formula 1 in combination with one or more, preferably one, compound of general formula 2b

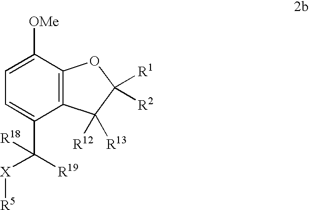

wherein

R¹ denotes hydrogen, methyl, ethyl or 4-pyridyl, or

together with R² denotes a butylene bridge;

R² denotes hydrogen, methyl, ethyl, or

together with R¹ denotes a butylene bridge, or

together with R¹³ denotes a single bond;

R⁵ denotes 3,5-dichloro-pyridin-4-yl or 4-pyridyl;

R¹² denotes hydrogen or methyl;

R¹³ denotes hydrogen or

together with R² denotes a single bond;

R¹⁸ denotes hydrogen or methyl;

R¹⁹ denotes hydrogen, methoxy, phenyl or CN;

X denotes —CH₂—, —S— or —NH—,

optionally in the form of the individual optical isomers, mixtures thereof or racemates and optionally in the form of the pharmacologically acceptable acid addition salts thereof.

More preferably, the present invention the present invention relates to a pharmaceutical composition which contains, in addition to an anticholinergic of formula 1 one or more, preferably one, compound of general formula 2b which is selected from the compounds according to Table 2.

TABLE 2
Particularly preferred compounds of formula 2b

| Example | R¹ | R² | R¹³ | R¹² | X | R¹⁸ | R¹⁹ | R⁵ |
|---|---|---|---|---|---|---|---|---|
| 45 | —Me | —Me | —H | —H | —CH₂— | —H | —H | 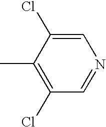 |
| 46 | —Me | —Me | —H | —H | —CH₂— | —H | —H | 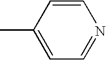 |
| 47 | —Me | —Me | —H | —H | —CH₂— | —H | —Ph | 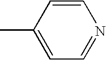 |
| 48 | —Me | —Me | —H | —H | —S— | —H | —H | 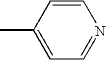 |
| 49 | —Me | —Me | —H | —H | —S— | —H | —Ph | 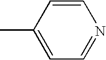 |
| 50 | —Et | —Et | —H | —H | —CH₂— | —H | —H | 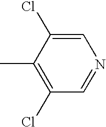 |
| 51 | —Et | —Et | —H | —H | —CH₂— | —H | —H | 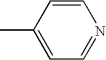 |
| 52 | —(CH₂)₄— | | —H | —H | —CH₂— | —H | —H | 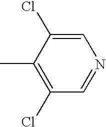 |
| 53 | —(CH₂)₄— | | —H | —H | —CH₂— | —H | —H | 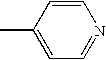 |
| 54 | —(CH₂)₅— | | —H | —H | —CH₂— | —H | —H | 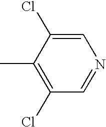 |
| 55 | —(CH₂)₅— | | —H | —H | —CH₂— | —H | —H | 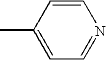 |
| 56 | —H | —H | —H | —Me | —CH₂— | —H | —H | 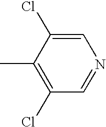 |
| 57 | —H | —H | —H | —Me | —CH₂— | —H | —H | 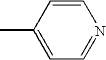 |
| 58 | —H | —H | —H | —Me | —CH₂— | —H | —Ph | 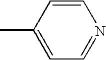 |
| 59 | —H | —H | —H | —Me | —S— | —H | —H | 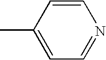 |
| 60 | —H | —H | —H | —Me | —S— | —H | —Ph | 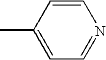 |
| 61 | —H | —H | —H | —Me | —NH— | —H | —H | 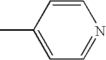 |
| 62 | —Me | —Me | —H | —H | —CH₂— | —H | —OMe | 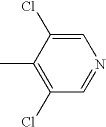 |
| 63 | —Me | —Me | —H | —H | —CH₂— | —H | —CN | 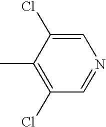 |

| | | | | | | | |
|---|---|---|---|---|---|---|---|
| 64 | —(CH₂)₄— | —H | —H | —CH₂— | —H | —CN | 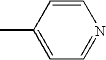 |
| 65 | —(CH₂)₄— | —H | —H | —CH₂— | —Me | —CN | 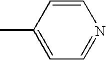 |

| | | | | | | | |
|---|---|---|---|---|---|---|---|
| 66 | 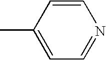 | single bond | —H | —CH₂— | —H | —Ph | 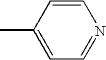 |

Compounds of general formula 2 wherein R³ denotes hydrogen and R⁴ denotes methoxy and wherein A denotes oxygen, B the group —C(R¹²)(R¹³)— and D the group —C(R^{19a})═Y—, have the general formula 2c

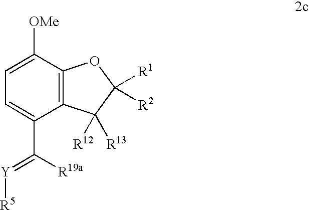

In a preferred aspect the present invention relates to a pharmaceutical composition, characterised in that it contains an anticholinergic of formula 1 in combination with one or more, preferably one, compound of general formula 2c

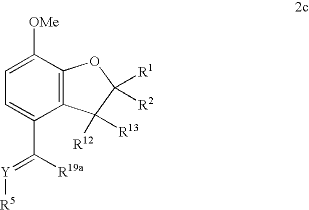

wherein

R¹ denotes hydrogen, methyl, ethyl, phenyl, 4-pyridyl, 2-pyridyl, or

together with R² denotes a butylene or pentylene bridge;

R² denotes hydrogen, methyl, ethyl, or

together with R¹ denotes a butylene or pentylene bridge, or

together with R¹³ denotes a single bond;

R⁵ denotes 3-methoxycarbonylphenyl, 4-methoxycarbonylphenyl, 3-carboxyphenyl, 4-carboxyphenyl, CN, —COOEt, 3,5-dichloro-pyridin-4-yl or 4-pyridyl;

R¹² denotes hydrogen or methyl;

R¹³ denotes hydrogen or

together with R² denotes a single bond;

R^{19a} denotes hydrogen, methyl or phenyl;

Y denotes CH, CCN, CCOOEt or CHCONH,

optionally in the form of the individual optical isomers, mixtures thereof or racemates and optionally in the form of the pharmacologically acceptable acid addition salts thereof.

More preferably, the present invention the present invention relates to a pharmaceutical composition which contains, in addition to an anticholinergic of formula 1, one or more, preferably one, compound of general formula 2c which is selected from the compounds according to Table 3.

TABLE 3
Particularly preferred compounds of formula 2c

| Example | R¹ | R² | R¹³ | R¹² | Y | R^{19a} | R⁵ |
|---|---|---|---|---|---|---|---|
| 67 | —Me | —Me | —H | —H | CH | —H | 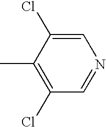 |
| 68 | —Me | —Me | —H | —H | CH | —H | 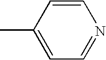 |
| 69 | —Me | —Me | —H | —H | CH | —Me | 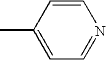 |
| 70 | —Me | —Me | —H | —H | CH | —Ph | 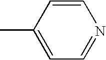 |
| 71 | —Et | —Et | —H | —H | CH | —H | 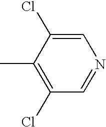 |
| 72 | —Et | —Et | —H | —H | CH | —H | 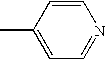 |
| 73 | —(CH₂)₄— | | —H | —H | CH | —H | 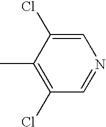 |
| 74 | —(CH₂)₄— | | —H | —H | CH | —H | 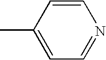 |
| 75 | —(CH₂)₄— | | —H | —H | CH | —Me | 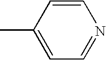 |
| 76 | —(CH₂)₅— | | —H | —H | CH | —H | 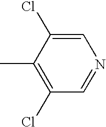 |
| 77 | —(CH₂)₅— | | —H | —H | CH | —H | 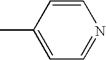 |
| 78 | —H | —H | —H | —Me | CH | —H | 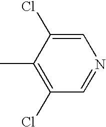 |
| 79 | —H | —H | —H | —Me | CH | —H | 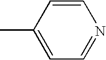 |
| 80 | —H | —H | —H | —Me | CH | —Ph | 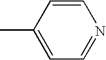 |
| 81 | —Ph | single bond | | —H | CH | —H | 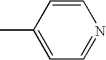 |
| 82 | 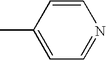 | single bond | | —H | CH | —H | 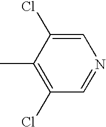 |
| 83 | 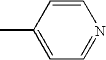 | single bond | | —H | CH | —H | 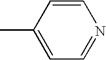 |
| 84 | 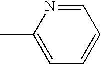 | single bond | | —H | CH | —H | 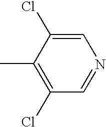 |
| 85 | 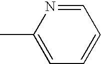 | single bond | | —H | CH | —H | 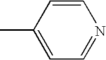 |
| 86 | —Me | —Me | —H | —H | CCN | —H | 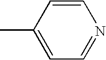 |
| 87 | —Me | —Me | —H | —H | CCO₂Et | —H | 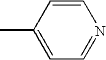 |
| 88 | —Me | —Me | —H | —H | CCN | —H | —CN |
| 89 | —Me | —Me | —H | —H | CCN | —H | —CO₂Et |

| | | | | | | |
|---|---|---|---|---|---|---|
| 90 | —(CH₂)₄— | —H | —H | CHCONH | —H | 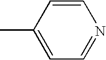 |
| 91 | —(CH₂)₄— | —H | —H | CHCONH | —H | 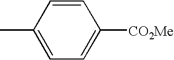 |
| 92 | —(CH₂)₄— | —H | —H | CHCONH | —H | 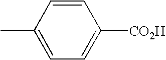 |
| 93 | —(CH₂)₄— | —H | —H | CHCONH | —H | 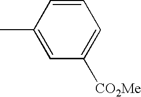 |
| 94 | —(CH₂)₄— | —H | —H | CHCONH | —H | 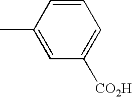 |

Compounds of general formula 2 wherein R³ denotes hydrogen and R⁴ denotes methoxy and wherein A denotes oxygen, B denotes the group —C(R¹²)(R¹³)— and D denotes the group —CO—CH₂—, have the general formula 2d

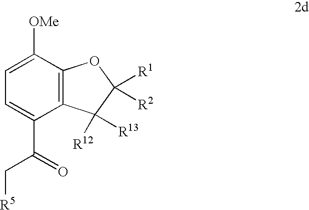

In a preferred aspect the present invention relates to a pharmaceutical composition, characterised in that it contains an anticholinergic of formula 1 in combination with one or more, preferably one, compound of general formula 2d

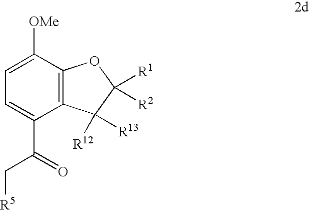

wherein

R¹ denotes hydrogen, methyl, ethyl, n-butyl, i-butyl, phenyl, 2-ethylphenyl, 2-1-propylphenyl, 4-pyridyl, 2-pyridyl, —CO-phenyl, CN, or

together with R² denotes a butylene or pentylene bridge;

R² denotes hydrogen, methyl, ethyl, or

together with R¹ denotes a butylene or pentylene bridge, or

together with R¹³ denotes a single bond or a butylene bridge;

R⁵ denotes phenyl, 3,5-dichloro-pyridin-4-yl or 4-pyridyl;

R¹² denotes hydrogen, methyl, phenyl or —CH₂—COR¹;

R¹³ denotes hydrogen or

together with R² denotes a single bond or a butylene bridge;

R^{x} denotes ethoxy,

optionally in the form of the individual optical isomers, mixtures thereof or racemates and optionally in the form of the pharmacologically acceptable acid addition salts thereof.

More preferably, the present invention relates to a pharmaceutical composition which contains, in addition to an anticholinergic of formula 1, one or more, preferably one, compound of general formula 2d which is selected from the compounds according to Table 4.

TABLE 4
Particularly preferred compounds of formula 2d

| Example | R¹ | R² | R¹³ | R¹² | R⁵ |
|---|---|---|---|---|---|
| 95 | —Me | —Me | —H | —H | 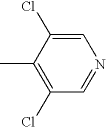 |
| 96 | —Me | —Me | —H | —H | 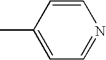 |
| 97 | —Et | —Et | —H | —H | 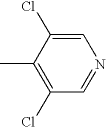 |
| 98 | —Et | —Et | —H | —H | 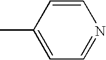 |
| 99 | —(CH₂)₄— | | —H | —H | 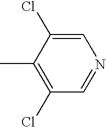 |
| 100 | —(CH₂)₄— | | —H | —H | 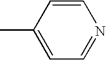 |
| 101 | —(CH₂)₅— | | —H | —H | 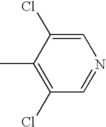 |
| 102 | —(CH₂)₅— | | —H | —H | 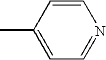 |
| 103 | —H | —H | —H | —Me | 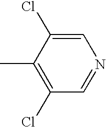 |
| 104 | —H | —H | —H | —Me | 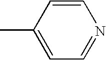 |

| | | | | |
|---|---|---|---|---|
| 105 | —H | —(CH₂)₄— | —H | 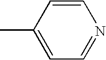 |
| 106 | —CN | single bond | —H | 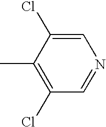 |
| 107 | —COphenyl | single bond | —H | —phenyl |
| 108 | —COphenyl | single bond | —H | 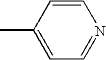 |
| 109 | -n-Bu | single bond | —H | 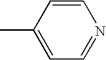 |
| 110 | -i-Bu | single bond | —H | 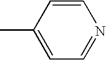 |
| 111 | —phenyl | single bond | —H | 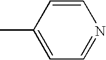 |
| 112 | 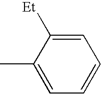 | single bond | —H | 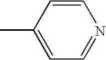 |
| 113 | 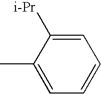 | single bond | —H | 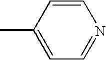 |
| 114 | 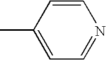 | single bond | —H | 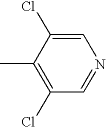 |
| 115 | 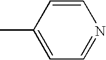 | single bond | —H | 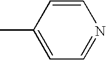 |
| 116 | 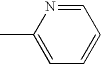 | single bond | —H | 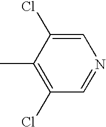 |
| 117 | 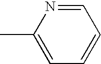 | single bond | —H | 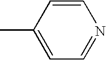 |
| 118 | —H | single bond | —Ph | 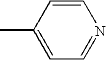 |
| 119 | —H | single bond | —CH₂—CO₂Et | 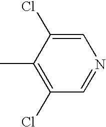 |
| 120 | —H | single bond | —CH₂—CO₂Et | 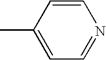 |

Compounds of general formula 2 wherein R³ denotes hydrogen and R⁴ denotes methoxy and wherein A denotes oxygen and B denotes the group —CH(R¹⁵)—CH(R¹⁷)— have the general formula 2e

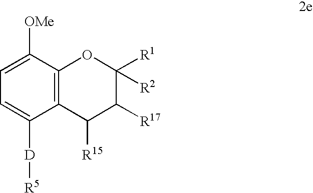

In a preferred aspect the present invention relates to a pharmaceutical composition, characterised in that it contains an anticholinergic of formula 1 in combination with one or more, preferably one, compound of general formula 2e

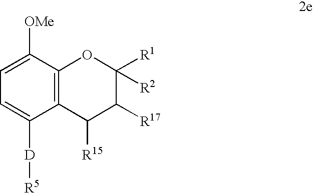

wherein

R¹ denotes methyl or

together with R² denotes a butylene or pentylene bridge;

R² denotes methyl or

together with R¹ denotes a butylene or pentylene bridge;

R⁵ denotes 3,5-dichloro-pyridin-4-yl or 4-pyridyl;

D denotes a group selected from —CONH—, —CO—CH₂— or —CH═CH—;

R¹⁵ denotes hydrogen or

together with R¹⁷ denotes a single bond;

R¹⁷ denotes hydrogen or

together with R¹⁵ denotes a single bond,

optionally in the form of the individual optical isomers, mixtures thereof or racemates and optionally in the form of the pharmacologically acceptable acid addition salts thereof.

More preferably, the present invention relates to a pharmaceutical composition which contains, in addition to an anticholinergic of formula 1, one or more, preferably one, compound of general formula 2e which is selected from the compounds according to Table 5.

TABLE 5
Particularly preferred compounds of formula 2e

| Example | R¹ | R² | R¹⁵ | R¹⁷ | D | R⁵ |
|---|---|---|---|---|---|---|
| 121 | —Me | —Me | single bond | | CONH | 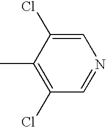 |
| 122 | —Me | —Me | —H | —H | CONH | 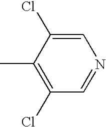 |

| 123 | —(CH₂)₄— | single bond | | CONH | 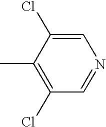 |
|---|---|---|---|---|---|
| 124 | —(CH₂)₄— | —H | —H | CONH | 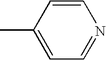 |
| 125 | —(CH₂)₄— | —H | —H | CH═CH | 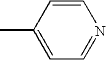 |
| 126 | —(CH₂)₅— | —H | —H | CH═CH | 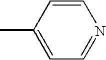 |
| 127 | —(CH₂)₄— | —H | —H | COCH₂ | 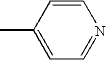 |
| 128 | —(CH₂)₅— | —H | —H | COCH₂ | 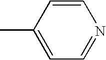 |

Compounds of general formula 2 wherein R³ denotes hydrogen and R⁴ denotes methoxy and wherein A denotes —CH₂— and B denotes oxygen and wherein R¹ and R² together form a butylene bridge, have the general formula 2f

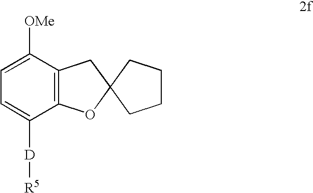

In a preferred aspect the present invention relates to a pharmaceutical composition, characterised in that it contains an anticholinergic of formula 1 in combination with one or more, preferably one, compound of general formula 2f

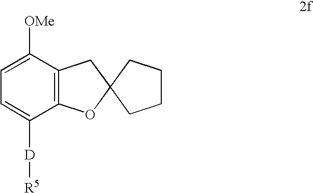

wherein

R⁵ denotes 3,5-dichloro-pyridin-4-yl or 4-pyridyl;

D denotes a group selected from —CONH—, —CO—CH₂— or —CH═CH—,

optionally in the form of the individual optical isomers, mixtures thereof or racemates and optionally in the form of the pharmacologically acceptable acid addition salts thereof.

More preferably, the present invention relates to a pharmaceutical composition which contains, in addition to an anticholinergic of formula 1, one or more, preferably one, compound of general formula 2f which is selected from the compounds according to Table 6.

TABLE 6
Particularly preferred compounds of formula 2f

| Example | D | R⁵ |
|---|---|---|
| 129 | CONH | 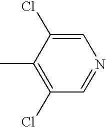 |
| 130 | CONH | 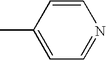 |
| 131 | CH═CH | 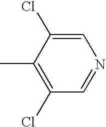 |
| 132 | COCH₂ | 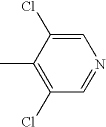 |
| 133 | COCH₂ | 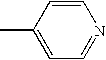 |

Compounds of general formula 2, wherein R³ denotes hydrogen and R⁴ denotes methoxy, wherein A and B denotes oxygen and R¹ and R² together form a butylene bridge, have the general formula 2g

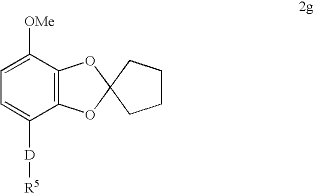

In a preferred aspect the present invention relates to a pharmaceutical composition, characterised in that it contains an anticholinergic of formula 1 combined with one or more, preferably one, compound of general formula 2g

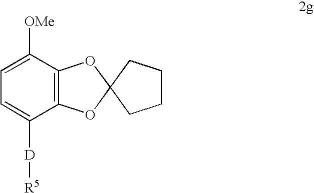

wherein

R⁵ may represent 3,5-dichloro-pyridin-4-yl or 4-pyridyl;

D may represent a group selected from —CH₂—CH₂—, —CH(Ph)—CH₂—, —CONH—, —CO—CH₂—, —CH═CH— or —C(Ph)═CH—,

optionally in the form of the individual optical isomers, mixtures thereof or racemates and optionally in the form of the pharmacologically acceptable acid addition salts thereof.

More preferably, the present invention relates to a pharmaceutical composition which contains, in addition to an anticholinergic of formula 1 one or more, preferably one, compound of general formula 2g which is selected from the compounds according to Table 7.

TABLE 7
Particularly preferred compounds of formula 2g

| Example | D | R⁵ |
|---|---|---|
| 134 | CONH | 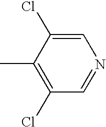 |
| 135 | CONH | 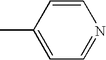 |
| 136 | CH₂CH₂ | 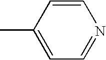 |
| 137 | CHPhCH₂ | 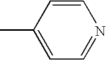 |
| 138 | CH═CH | 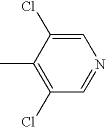 |
| 139 | CPh═CH | 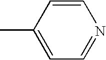 |
| 140 | COCH₂ | 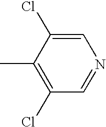 |
| 141 | COCH₂ | 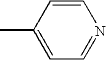 |

Compounds of general formula 2 wherein R³ denotes hydrogen and R⁴ denotes methoxy, wherein A denotes oxygen and B denotes —CH₂—, and R¹ and R² together form a butylene bridge and wherein the group -D-R⁵ denotes the group W, have the general formula 2h

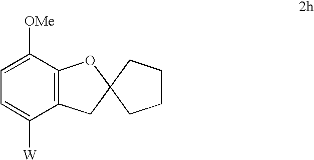

In a preferred aspect the present invention relates to a pharmaceutical composition, characterised in that it contains an anticholinergic of formula 1 in combination with a compound of general formula 2h

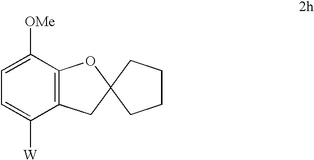

wherein

W denotes a group selected from among

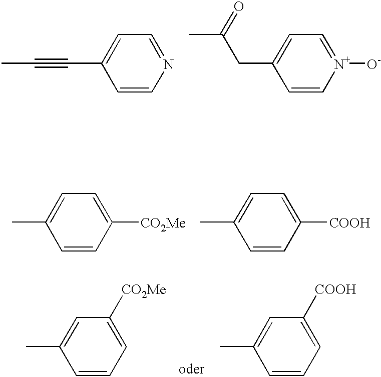

optionally in the form of the individual optical isomers, mixtures thereof or racemates and optionally in the form of the pharmacologically acceptable acid addition salts thereof.

More preferably, the present invention relates to a pharmaceutical composition which contains, in addition to an anticholinergic of formula 1 one or more, preferably one, compound of general formula 2h which is selected from the compounds according to Table 8.

TABLE 8
Particularly preferred compounds of formula 2h

| Example | W |
|---|---|
| 142 | 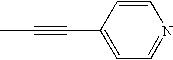 |
| 143 | 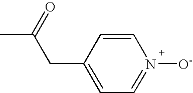 |
| 144 | 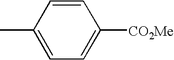 |
| 145 | 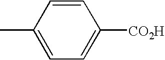 |
| 146 | 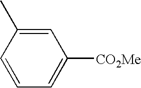 |
| 147 | 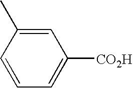 |

Any reference to the abovementioned compounds 2 also includes within the scope of the present invention a reference to any pharmacologically acceptable acid addition salts thereof which may exist. By physiologically acceptable acid addition salts which may be formed from 2 are meant according to the invention pharmaceutically acceptable salts which are selected from the salts of hydrochloric acid, hydrobromic acid, sulphuric acid, phosphoric acid, methanesulphonic acid, acetic acid, fumaric acid, succinic acid, lactic acid, citric acid, tartaric acid or maleic acid. According to the invention, the salts of the compounds 2 selected from among the acetate, hydrochloride, hydrobromide, sulphate, phosphate and methanesulphonate are preferred.

Any reference to compounds 1 within the scope of the invention includes a reference to the solvates and hydrates optionally formed by 1.

The pharmaceutical combinations of 1 and 2 according to the invention are preferably administered by inhalation. Suitable inhalable powders packed into suitable capsules may be administered using suitable powder inhalers. Alternatively, the drug may be inhaled by the application of suitable inhalation aerosols. These also include inhalation aerosols which contain HFA134a (also known as TG134a), HFA227 (also known as TG227) or a mixture thereof as propellant gas, for example. The drug may also be inhaled using suitable solutions of the pharmaceutical combination consisting of 1 and 2.

In another aspect the present invention relates to a pharmaceutical composition which contains one or more salts 1 and one or more compounds 2, optionally in the form of their solvates or hydrates. The active substances may be combined in a single preparation or contained in two separate formulations. Pharmaceutical compositions which contain the active substances 1 and 2 in a single preparation are preferred according to the invention.

In another aspect the present invention relates to a pharmaceutical composition which contains, in addition to therapeutically effective quantities of 1 and 2, a pharmaceutically acceptable carrier or excipient. In another aspect the present invention relates to a pharmaceutical composition which does not contain any pharmaceutically acceptable excipient in addition to therapeutically effective quantities of 1 and 2.

The present invention also relates to the use of 1 and 2 for preparing a pharmaceutical composition containing therapeutically effective quantities of 1 and 2 for treating inflammatory and/or obstructive diseases of the respiratory tract, particularly asthma or chronic obstructive pulmonary disease (COPD), as well as complications thereof such as pulmonary hypertension, as well as allergic and non-allergic rhinitis.

The present invention also relates to the use of a pharmaceutical formulation containing one or more, preferably one, compound of formula 1 for preparing a pharmaceutical composition for treating inflammatory and/or obstructive diseases of the respiratory tract, particularly asthma or chronic obstructive pulmonary disease (COPD), as well as complications thereof such as pulmonary hypertension, as well as allergic and non-allergic rhinitis, characterised in that the pharmaceutical formulation contains one or more, preferably one, compound of formula 2.

The present invention also relates to the simultaneous or successive use of therapeutically effective doses of the combination of the above pharmaceutical compositions 1 and 2 for treating inflammatory and/or obstructive diseases of the respiratory tract, particularly asthma or chronic obstructive pulmonary disease (COPD), as well as complications thereof such as pulmonary hypertension, as well as allergic and non-allergic rhinitis, by simultaneous or successive administration.

In the active substance combinations of 1 and 2 according to the invention, ingredients 1 and 2 may be present in the form of their enantiomers, mixtures of enantiomers or in the form of racemates.

The proportions in which the active substances 1 and 2 may be used in the active substance combinations according to the invention are variable. Active substances 1 and 2 may possibly be present in the form of their solvates or hydrates. Depending on the choice of the compounds 1 and 2, the weight ratios which may be used within the scope of the present invention vary on the basis of the different molecular weights of the various compounds and their different potencies. As a rule, the pharmaceutical combinations according to the invention may contain compounds 1 and 2 in ratios by weight ranging from 1:300 to 80:1, preferably from 1:250 to 70:1. In the particularly preferred pharmaceutical combinations which contain compound 1 in the form of its bromide, the weight ratios of 1 to 2 are preferably in the range from 1:150 to 60:1, more preferably from 1:50 to 40:1.

For example and without restricting the scope of the invention thereto, preferred combinations of 1 and 2 according to the invention may contain the cation 1′ and a compound of formula 2 in the following weight ratios: 1:50; 1:49; 1:48; 1:47; 1:46; 1:45; 1:44; 1:43; 1:42; 1:41; 1:40; 1:39; 1:38; 1:37; 1:36; 1:35; 1:34; 1:33; 1:32; 1:31; 1:30; 1:29; 1:28; 1:27; 1:26; 1:25; 1:24; 1:23; 1:22; 1:21; 1:20; 1:19; 1:18; 1:17; 1:16; 1:15; 1:14; 1:13; 1:12; 1:11; 1:10; 1:9; 1:8; 1:7; 1:6; 1:5; 1:4; 1:3; 1:2; 1:1; 2:1; 3:1; 4:1; 5:1; 6:1; 7:1; 8:1; 9:1; 10:1; 11:1; 12:1; 13:1; 14:1; 15:1; 16:1; 17:1; 18:1; 19:1; 20:1.

The pharmaceutical compositions according to the invention containing the combinations of 1 and 2 are normally used so that 1 and 2 are present together in doses from 0.01 to 10000 μg, preferably from 0.1 to 2000 μg, more preferably from 1 to 1500 μg per single dose. For example combinations of 1 and 2 according to the invention contain an amount of cation 1′ and compound of formula 2 such that the total dosage per single dose is 100 μg, 105 μg, 110 μg, 115 μg, 120 μg, 125 μg, 130 μg, 135 μg, 140 μg, 145 μg, 150 μg, 155 μg, 160 μg, 165 μg, 170 μg, 175 μg, 180 μg, 185 μg, 190 μg, 195 μg, 200 μg, 205 μg, 210 μg, 215 μg, 220 μg, 225 μg, 230 μg, 235 μg, 240 μg, 245 μg, 250 μg, 255 μg, 260 μg, 265 μg, 270 μg, 275 μg, 280 μg, 285 μg, 290 μg, 295 μg, 300 μg, 305 μg, 310 μg, 315 μg, 320 μg, 325 μg, 330 μg, 335 μg, 340 μg, 345 μg, 350 μg, 355 μg, 360 μg, 365 μg, 370 μg, 375 μg, 380 μg, 385 μg, 390 μg, 395 μg, 400 μg, 405 μg, 410 μg, 415 μg, 420 μg, 425 μg, 430 μg, 435 μg, 440 μg, 445 μg, 450 μg, 455 μg, 460 μg, 465 μg, 470 μg, 475 μg, 480 μg, 485 μg, 490 μg, 495 μg, 500 μg, 505 μg, 510 μg, 515 μg, 520 μg, 525 μg, 530 μg, 535 μg, 540 μg, 545 μg, 550 μg, 555 μg, 560 μg, 565 μg, 570 μg, 575 μg, 580 μg, 585 μg, 590 μg, 595 μg, 600 μg, 605 μg, 610 μg, 615 μg, 620 μg, 625 μg, 630 μg, 635 μg, 640 μg, 645 μg, 650 μg, 655 μg, 660 μg, 665 μg, 670 μg, 675 μg, 680 μg, 685 μg, 690 μg, 695 μg, 700 μg, 705 μg, 710 μg, 715 μg, 720 μg, 725 μg, 730 μg, 735 μg, 740 μg, 745 μg, 750 μg, 755 μg, 760 μg, 765 μg, 770 μg, 775 μg, 780 μg, 785 μg, 790 μg, 795 μg, 800 μg, 805 μg, 810 μg, 815 μg, 820 μg, 825 μg, 830 μg, 835 μg, 840 μg, 845 μg, 850 μg, 855 μg, 860 μg, 865 μg, 870 μg, 875 μg, 880 μg, 885 μg, 890 μg, 895 μg, 900 μg, 905 μg, 910 μg, 915 μg, 920 μg, 925 μg, 930 μg, 935 μg, 940 μg, 945 μg, 950 μg, 955 μg, 960 μg, 965 μg, 970 μg, 975 μg, 980 μg, 985 μg, 990 μg, 995 μg, 1000 μg, 1005 μg, 1010 μg, 1015 μg, 1020 μg, 1025 μg, 1030 μg, 1035 μg, 1040 μg, 1045 μg, 1050 μg, 1055 μg, 1060 μg, 1065 μg, 1070 μg, 1075 μg, 1080 μg, 1085 μg, 1090 μg, 1095 μg, 1100 μg or the like. These proposed dosages per single dose are not to be regarded as being restricted to the numerical values explicitly mentioned but are merely disclosed by way of example. Obviously, dosages which fluctuate around these values within a range of about +/− 2.5 μg are also covered by the values mentioned by way of example. In these dosage ranges the active substances 1′ and 2 may be present in the weight ratios described above.

For example and without restricting the scope of the invention thereto, the combinations of 1 and 2 according to the invention may contain an amount of cation 1′ and compound of formula 2 such that 8.3 μg of 1′ and 25 μg of 2, 8.3 μg of 1′ and 50 μg of 2, 8.3 μg of 1′ and 100 μg of 2, 8.3 μg of 1′ and 200 μg of 2, 8.3 μg of 1′ and 300 μg of 2, 8.3 μg of 1′ and 400 μg of 2, 8.3 μg of 1′ and 500 μg of 2, 8.3 μg of 1′ and 600 μg of 2, 8.3 μg of 1′ and 700 μg of 2, 8.3 μg of 1′ and 800 μg of 2, 8.3 μg of 1′ and 900 μg of 2, 8.3 μg of 1′ and 1000 μg of 2, 16.5 μg of 1′ and 25 μg of 2, 16.5 μg of 1′ and 50 μg of 2, 16.5 μg of 1′ and 100 μg of 2, 16.5 μg of 1′ and 200 μg of 2, 16.5 μg of 1′ and 300 μg of 2, 16.5 μg of 1′ and 400 μg of 2, 16.5 μg of 1′ and 500 μg of 2, 16.5 μg of 1′ and 600 μg of 2, 16.5 μg of 1′ and 700 μg of 2, 16.5 μg of 1′ and 800 μg of 2, 16.5 μg of 1′ and 900 μg of 2, 16.5 μg of 1′ and 1000 μg of 2, 33 μg of 1′ and 25 μg of 2, 33 μg of 1′ and 50 μg of 2, 33 μg of 1′ and 100 μg of 2, 33 μg of 1′ and 200 μg of 2, 33 μg of 1′ and 300 μg of 2, 33 μg of 1′ and 400 μg of 2, 33 μg of 1′ and 500 μg of 2, 33 μg of 1′ and 600 μg of 2, 33 μg of 1′ and 700 μg of 2, 33 μg of 1′ and 800 μg of 2, 33 μg of 1′ and 900 μg of 2, 33 μg of 1′ and 1000 μg of 2, 49.5 μg of 1′ and 25 μg of 2, 49.5 μg of 1′ and 50 μg of 2, 49.5 μg of 1′ and 100 μg of 2, 49.5 μg of 1′ and 200 μg of 2, 49.5 μg of 1′ and 300 μg of 2, 49.5 μg of 1′ and 400 μg of 2, 49.5 μg of 1′ and 500 μg of 2, 49.5 μg of 1′ and 600 μg of 2, 49.5 μg of 1′ and 700 μg of 2, 49.5 μg of 1′ and 800 μg of 2, 49.5 μg of 1′ and 900 μg of 2, 49.5 μg of 1′ and 1000 μg of 2, 82.6 μg of 1′ and 25 μg of 2, 82.6 μg of 1′ and 50 μg of 2, 82.6 μg of 1′ and 100 μg of 2, 82.6 μg of 1′ and 200 μg of 2, 82.6 μg of 1′ and 300 μg of 2, 82.6 μg of 1′ and 400 μg of 2, 82.6 μg of 1′ and 500 μg of 2, 82.6 μg of 1′ and 600 μg of 2, 82.6 μg of 1′ and 700 μg of 2, 82.6 μg of 1′ and 800 μg of 2, 82.6 μg of 1′ and 900 μg of 2, 82.6 μg of 1′ and 1000 μg of 2, 165.1 μg of 1′ and 25 μg of 2, 165.1 μg of 1′ and 50 μg of 2, 165.1 μg of 1′ and 100 μg of 2, 165.1 μg of 1′ and 200 μg of 2, 165.1 μg of 1′ and 300 μg of 2, 165.1 μg of 1′ and 400 μg of 2, 165.1 μg of 1′ and 500 μg of 2, 165.1 μg of 1′ and 600 μg of 2, 165.1 μg of 1′ and 700 μg of 2, 165.1 μg of 1′ and 800 μg of 2, 165.1 μg of 1′ and 900 μg of 2, 165.1 μg of 1′ and 1000 μg of 2, 206, 4 μg of 1′ and 25 μg of 2, 206.4 μg of 1′ and 50 μg of 2, 206.4 μg of 1′ and 100 μg of 2, 206.4 μg of 1′ and 200 μg of 2, 206.4 μg of 1′ and 300 μg of 2, 206.4 μg of 1′ and 400 μg of 2, 206.4 μg of 1′ and 500 μg of 2, 206.4 μg of 1′ and 600 μg of 2, 206.4 μg of 1′ and 700 μg of 2, 206.4 μg of 1′ and 800 μg of 2, 206.4 μg of 1′ and 900 μg of 2, 206.4 μg of 1′ and 1000 μg of 2, 412.8 μg of 1′ and 25 μg of 2, 412.8 μg of 1′ and 50 μg of 2, 412.8 μg of 1′ and 100 μg of 2, 412.8 μg of 1′ and 200 μg of 2, 412.8 μg of 1′ and 300 μg of 2, 412.8 μg of 1′ and 400 μg of 2, 412.8 μg of 1′ and 500 μg of 2 or 412.8 μg of 1′ and 600 μg of 2, 412.8 μg of 1′ and 700 μg of 2, 412.8 μg of 1′ and 800 μg of 2, 412.8 μg of 1′ and 900 μg of 2, 412.8 μg of 1′ and 1000 μg of 2 are administered per single dose.

If the active substance combination wherein 1 denotes the bromide is used as the preferred combination of 1 and 2 according to the invention, the quantities of active substances 1′ and 2 administered per single dose as specified by way of example correspond to the following quantities of 1 and 2 administered per single dose: 10 μg of 1 and 25 μg of 2, 10 μg of 1 and 50 μg of 2, 10 μg of 1 and 100 μg of 2, 10 μg of 1 and 200 μg of 2, 10 μg of 1 and 300 μg of 2, 10 μg of 1 and 400 μg of 2, 10 μg of 1 and 500 μg of 2, 10 μg of 1 and 600 μg of 2, 10 μg of 1 and 700 μg of 2, 100 μg of 1 and 800 μg of 2, 10 μg of 1 and 900 μg of 2, 10 μg of 1 and 100 μg of 2, 20 μg of 1 and 25 μg of 2, 20 μg of 1 and 50 μg of 2, 20 μg of 1 and 100 μg of 2, 20 μg of 1 and 200 μg of 2, 20 μg of 1 and 300 μg of 2, 20 μg of 1 and 400 μg of 2, 20 μg of 1 and 500 μg of 2, 20 μg of 1 and 600 μg of 2, 20 μg of 1 and 700 μg of 2, 20 μg of 1 and 800 μg of 2, 20 μg of 1 and 900 μg of 2, 20 μg of 1 and 1000 μg of 2, 40 μg of 1 and 25 μg of 2, 40 μg of 1 and 50 μg of 2, 40 μg of 1 and 100 μg of 2, 40 μg of 1 and 200 μg of 2, 40 μg of 1 and 300 μg of 2, 40 μg of 1 and 400 μg of 2, 40 μg of 1 and 500 μg of 2, 40 μg of 1 and 600 μg of 2, 40 μg of 1 and 700 μg of 2, 40 μg of 1 and 800 μg of 2, 40 μg of 1 and 900 μg of 2, 40 μg of 1 and 1000 μg of 2, 60 μg of 1 and 25 μg of 2, 60 μg of 1 and 50 μg of 2, 60 μg of 1 and 100 μg of 2, 60 μg of 1 and 200 μg of 2, 60 μg of 1 and 300 μg of 2, 60 μg of 1 and 400 μg of 2, 60 μg of 1 and 500 μg of 2, 60 μg of 1 and 600 μg of 2, 60 μg of 1 and 700 μg of 2, 60 μg of 1 and 800 μg of 2, 60 μg of 1 and 900 μg of 2, 60 μg of 1 and 1000 μg of 2, 100 μg of 1 and 25 μg of 2, 100 μg of 1 and 50 μg of 2, 100 μg of 1 and 100 μg of 2, 100 μg of 1 and 200 μg of 2, 100 μg of 1 and 300 μg of 2, 100 μg of 1 and 400 μg of 2, 100 μg of 1 and 500 μg of 2, 100 μg of 1 and 600 μg of 2, 100 μg of 1 and 700 μg of 2, 100 μg of 1 and 800 μg of 2, 100 μg of 1 and 900 μg of 2, 100 μg of 1 and 1000 μg of 2, 200 μg of 1 and 25 μg of 2, 200 μg of 1 and 50 μg of 2, 200 μg of 1 and 100 μg of 2, 200 μg of 1 and 200 μg of 2, 200 μg of 1 and 300 μg of 2, 200 μg of 1 and 400 μg of 2, 200 μg of 1 and 500 μg of 2, 200 μg of 1 and 600 μg of 2, 200 μg of 1 and 700 μg of 2, 200 μg of 1 and 800 μg of 2, 200 μg of 1 and 900 μg of 2, 200 μg of 1 and 1000 μg of 2, 250 μg of 1 and 25 μg of 2, 250 μg of 1 and 50 μg of 2, 250 μg of 1 and 100 μg of 2, 250 μg of 1 and 200 μg of 2, 250 μg of 1 and 300 μg of 2, 250 μg of 1 and 400 μg of 2, 250 μg of 1 and 500 μg of 2, 250 μg of 1 and 600 μg of 2, 250 μg of 1 and 700 μg of 2, 250 μg of 1 and 800 μg of 2, 250 μg of 1 and 900 μg of 2, 250 μg of 1 and 1000 μg of 2, 500 μg of 1 and 25 μg of 2, 500 μg of 1 and 50 μg of 2, 500 μg of 1 and 100 μg of 2, 500 μg of 1 and 200 μg of 2, 500 μg of 1 and 300 μg of 2, 500 μg of 1 and 400 μg of 2, 500 μg of 1 and 500 μg of 2, 500 μg of 1 and 600 μg of 2, 500 μg of 1 and 700 μg of 2, 500 μg of 1 and 800 μg of 2, 500 μg of 1 and 900 μg of 2 or 500 μg of 1 and 1000 μg of 2.

The active substance combinations of 1 and 2 according to the invention are preferably administered by inhalation. For this purpose, ingredients 1 and 2 have to be made available in forms suitable for inhalation. Inhalable preparations include inhalable powders, propellant-containing metering aerosols or propellant-free inhalable solutions. Inhalable powders according to the invention containing the combination of active substances 1 and 2 may consist of the active substances on their own or of a mixture of the active substances with physiologically acceptable excipients. Within the scope of the present invention, the term propellant-free inhalable solutions also includes concentrates or sterile inhalable solutions ready for use. The preparations according to the invention may contain the combination of active substances 1 and 2 either together in one formulation or in two separate formulations. These formulations which may be used within the scope of the present invention are described in more detail in the next part of the specification.

A) Inhalable Powder Containing the Combinations of Active Substances 1 and 2 According to the Invention:

The inhalable powders according to the invention may contain 1 and 2 either on their own or in admixture with suitable physiologically acceptable excipients.

If the active substances 1 and 2 are present in admixture with physiologically acceptable excipients, the following physiologically acceptable excipients may be used to prepare these inhalable powders according to the invention: monosaccharides (e.g. glucose or arabinose), disaccharides (e.g. lactose, saccharose, maltose, trehalose), oligo- and polysaccharides (e.g. dextran), polyalcohols (e.g. sorbitol, mannitol, xylitol), salts (e.g. sodium chloride, calcium carbonate) or mixtures of these excipients. Preferably, mono- or disaccharides are used, while the use of lactose or glucose is preferred, particularly, but not exclusively, in the form of their hydrates. For the purposes of the invention, lactose is the particularly preferred excipient, while lactose monohydrate is most particularly preferred.

Within the scope of the inhalable powders according to the invention the excipients have a maximum average particle size of up to 250 μm, preferably between 10 and 150 μm, most preferably between 15 and 80 μm. It may sometimes seem appropriate to add finer excipient fractions with an average particle size of 1 to 9 μm to the excipient mentioned above. These finer excipients are also selected from the group of possible excipients listed hereinbefore. Finally, in order to prepare the inhalable powders according to the invention, micronised active substance 1 and 2, preferably with an average particle size of 0.5 to 10 μm, more preferably from 1 to 5 μm, is added to the excipient mixture. Processes for producing the inhalable powders according to the invention by grinding and micronising and by finally mixing the ingredients together are known from the prior art. The inhalable powders according to the invention may be prepared and administered either in the form of a single powder mixture which contains both 1 and 2 or in the form of separate inhalable powders which contain only 1 or 2.

The inhalable powders according to the invention may be administered using inhalers known from the prior art. Inhalable powders according to the invention which contain a physiologically acceptable excipient in addition to 1 and 2 may be administered, for example, by means of inhalers which deliver a single dose from a supply using a measuring chamber as described in U.S. Pat. No. 4,570,630A, or by other means as described in DE 36 25 685 A. Preferably, the inhalable powders according to the invention which contain physiologically acceptable excipients in addition to 1 and 2 are packed into capsules which are used in inhalers as described, for example, in WO 94/28958.

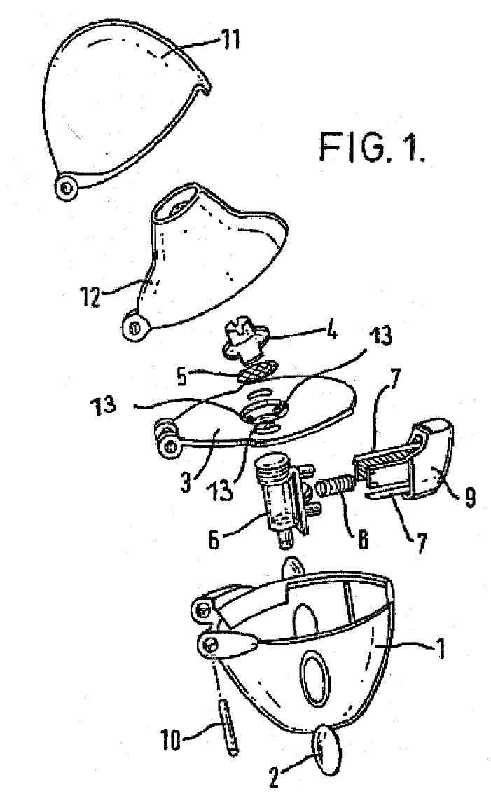

A particularly preferred inhaler for using the pharmaceutical combination according to the invention where the inhalable powder is placed in a capsule is shown in FIG. 1.

This inhaler for inhaling powdered pharmaceutical compositions from capsules is characterised by a housing 1 containing two windows 2, a deck 3 in which there are air inlet ports and which is provided with a screen 5 secured via a screen housing 4, an inhalation chamber 6 connected to the deck 3 on which there is a push button 9 provided with two sharpened pins 7 and movable counter to a spring 8, and a mouthpiece 12 which is connected to the housing 1, the deck 3 and a cover 11 via a spindle 10 to enable it to be flipped open or shut and airholes 13 for adjusting the flow resistance.

If the inhalable powders according to the invention are packed into capsules for the preferred use described above, the quantities packed into each capsule should be 1 to 30 mg, preferably 3 to 20 mg, more particularly 5 to 10 mg of inhalable powder per capsule. These capsules contain, according to the invention, either together or separately, the doses of 1 or 1′ and 2 mentioned hereinbefore for each single dose.

B) Propellant Gas-Driven Inhalation Aerosols Containing the Combinations of Active Substances 1 and 2:

Inhalation aerosols containing propellant gas according to the invention may contain substances 1 and 2 dissolved in the propellant gas or in dispersed form. 1 and 2 may be present in separate formulations or in a single preparation, in which 1 and 2 are either both dissolved, both dispersed or only one component is dissolved and the other is dispersed. The propellant gases which may be used to prepare the inhalation aerosols according to the invention are known from the prior art. Suitable propellant gases are selected from among hydrocarbons such as n-propane, n-butane or isobutane and halohydrocarbons such as fluorinated derivatives of methane, ethane, propane, butane, cyclopropane or cyclobutane. The propellant gases mentioned above may be used on their own or in mixtures thereof. Particularly preferred propellant gases are halogenated alkane derivatives selected from TG11, TG12, TG134a (1,1,1,2-tetrafluoroethane) and TG227 (1,1,1,2,3,3,3-heptafluoropropane) and mixtures thereof, of which the propellant gases TG134a, TG227 and mixtures thereof are preferred.

The propellant-driven inhalation aerosols according to the invention may also contain other ingredients such as co-solvents, stabilisers, surfactants, antioxidants, lubricants, preservatives and pH adjusters. All these ingredients are known in the art.

The inhalation aerosols containing propellant gas according to the invention may contain up to 5 wt.-% of active substance 1 and/or 2. Aerosols according to the invention contain, for example, 0.002 to 5 wt.-%, 0.01 to 3 wt.-%, 0.015 to 2 wt.-%, 0.1 to 2 wt.-%, 0.5 to 2 wt.-% or 0.5 to 1 wt.-% of active substance 1 and/or 2.

If the active substances 1 and/or 2 are present in dispersed form, the particles of active substance preferably have an average particle size of up to 10 μm, preferably from 0.1 to 6 μm, more preferably from 1 to 5 μm.

The propellant-driven inhalation aerosols according to the invention mentioned above may be administered using inhalers known in the art (MDIs=metered dose inhalers). Accordingly, in another aspect, the present invention relates to pharmaceutical compositions in the form of propellant-driven aerosols as hereinbefore described combined with one or more inhalers suitable for administering these aerosols. In addition, the present invention relates to inhalers which are characterised in that they contain the propellant gas-containing aerosols described above according to the invention. The present invention also relates to cartridges which are fitted with a suitable valve and can be used in a suitable inhaler and which contain one of the above-mentioned propellant gas-containing inhalation aerosols according to the invention. Suitable cartridges and methods of filling these cartridges with the inhalable aerosols containing propellant gas according to the invention are known from the prior art.

C) Propellant-Free Inhalable Solutions or Suspensions Containing the Combinations of Active Substances 1 and 2 According to the Invention:

Propellant-free inhalable solutions and suspensions according to the invention contain, for example, aqueous or alcoholic, preferably ethanolic solvents, optionally ethanolic solvents mixed with aqueous solvents. If aqueous/ethanolic solvent mixtures are used the relative proportion of ethanol compared with water is not limited but the maximum is up to 70 percent by volume, more particularly up to 60 percent by volume and most preferably up to 30 percent by volume. The remainder of the volume is made up of water. The solutions or suspensions containing 1 and 2, separately or together, are adjusted to a pH of 2 to 7, preferably 2 to 5, using suitable acids. The pH may be adjusted using acids selected from inorganic or organic acids. Examples of suitable inorganic acids include hydrochloric acid, hydrobromic acid, nitric acid, sulphuric acid and/or phosphoric acid. Examples of particularly suitable organic acids include ascorbic acid, citric acid, malic acid, tartaric acid, maleic acid, succinic acid, fumaric acid, acetic acid, formic acid and/or propionic acid etc. Preferred inorganic acids are hydrochloric and sulphuric acids. It is also possible to use the acids which have already formed an acid addition salt with one of the active substances. Of the organic acids, ascorbic acid, fumaric acid and citric acid are preferred. If desired, mixtures of the above acids may be used, particularly in the case of acids which have other properties in addition to their acidifying qualities, e.g. as flavourings, antioxidants or complexing agents, such as citric acid or ascorbic acid, for example. According to the invention, it is particularly preferred to use hydrochloric acid to adjust the pH.

According to the invention, the addition of editic acid (EDTA) or one of the known salts thereof, sodium editate, as stabiliser or complexing agent is unnecessary in the present formulation. Other embodiments may contain this compound or these compounds. In a preferred embodiment the content based on sodium editate is less than 100 mg/100 ml, preferably less than 50 mg/100 ml, more preferably less than 20 mg/100 ml. Generally, inhalable solutions in which the content of sodium editate is from 0 to 10 mg/100 ml are preferred.

Co-solvents and/or other excipients may be added to the propellant-free inhalable solutions according to the invention. Preferred co-solvents are those which contain hydroxyl groups or other polar groups, e.g. alcohols—particularly isopropyl alcohol, glycols—particularly propyleneglycol, polyethyleneglycol, polypropyleneglycol, glycolether, glycerol, polyoxyethylene alcohols and polyoxyethylene fatty acid esters. The terms excipients and additives in this context denote any pharmacologically acceptable substance which is not an active substance but which can be formulated with the active substance or substances in the physiologically suitable solvent in order to improve the qualitative properties of the active substance formulation. Preferably, these substances have no pharmacological effect or, in connection with the desired therapy, no appreciable or at least no undesirable pharmacological effect. The excipients and additives include, for example, surfactants such as soya lecithin, oleic acid, sorbitan esters, such as polysorbates, polyvinylpyrrolidone, other stabilisers, complexing agents, antioxidants and/or preservatives which guarantee or prolong the shelf life of the finished pharmaceutical formulation, flavourings, vitamins and/or other additives known in the art. The additives also include pharmacologically acceptable salts such as sodium chloride as isotonic agents.

The preferred excipients include antioxidants such as ascorbic acid, for example, provided that it has not already been used to adjust the pH, vitamin A, vitamin E, tocopherols and similar vitamins and provitamins occurring in the human body.

Preservatives may be used to protect the formulation from contamination with pathogens. Suitable preservatives are those which are known in the art, particularly cetyl pyridinium chloride, benzalkonium chloride or benzoic acid or benzoates such as sodium benzoate in the concentration known from the prior art. The preservatives mentioned above are preferably present in concentrations of up to 50 mg/100 ml, more preferably between 5 and 20 mg/100 ml.

Preferred formulations contain, in addition to the solvent water and the combination of active substances 1 and 2, only benzalkonium chloride and sodium editate. In another preferred embodiment, no sodium editate is present.

The propellant-free inhalable solutions according to the invention are administered in particular using inhalers of the kind which are capable of nebulising a small amount of a liquid formulation in the therapeutic dose within a few seconds to produce an aerosol suitable for therapeutic inhalation. Within the scope of the present invention, preferred inhalers are those in which a quantity of less than 100 μL, preferably less than 50 μL, more preferably between 20 and 30 μL of active substance solution can be nebulised in preferably one spray action to form an aerosol with an average particle size of less than 20 μm, preferably less than 10 μm, in such a way that the inhalable part of the aerosol corresponds to the therapeutically effective quantity.

An apparatus of this kind for propellant-free delivery of a metered quantity of a liquid pharmaceutical composition for inhalation is described for example in International Patent Application WO 91/14468 and also in WO 97/12687 (cf. in particular FIGS. 6a and 6b). The nebulisers (devices) described therein are known by the name Respimat®, developed for Boehringer Ingelheim International GmbH, Binger Strasse 173, 55216 Ingelheim, Federal Republic of Germany.

This nebuliser (Respimat®) can advantageously be used to produce the inhalable aerosols according to the invention containing the combination of active substances 1 and 2. Because of its cylindrical shape and handy size of less than 9 to 15 cm long and 2 to 4 cm wide, this device can be carried at all times by the patient. The nebuliser sprays a defined volume of pharmaceutical formulation using high pressures through small nozzles so as to produce inhalable aerosols.

The preferred atomiser essentially consists of an upper housing part, a pump housing, a nozzle, a locking mechanism, a spring housing, a spring and a storage container, characterised by

a pump housing which is secured in the upper housing part and which comprises at one end a nozzle body with the nozzle or nozzle arrangement,

a hollow plunger with valve body,

a power takeoff flange in which the hollow plunger is secured and which is located in the upper housing part,

a locking mechanism situated in the upper housing part,

a spring housing with the spring contained therein, which is rotatably mounted on the upper housing part by means of a rotary bearing,

a lower housing part which is fitted onto the spring housing in the axial direction.

The hollow plunger with valve body corresponds to a device disclosed in WO 97/12687. It projects partially into the cylinder of the pump housing and is axially movable within the cylinder. Reference is made in particular to FIGS. 1 to 4, especially FIG. 3, and the relevant parts of the description. The hollow plunger with valve body exerts a pressure of 5 to 60 Mpa (about 50 to 600 bar), preferably 10 to 60 Mpa (about 100 to 600 bar) on the fluid, the measured amount of active substance solution, at its high pressure end at the moment when the spring is actuated. Volumes of 10 to 50 microlitres are preferred, while volumes of 10 to 20 microlitres are particularly preferred and a volume of 15 microlitres per spray is most particularly preferred.

The valve body is preferably mounted at the end of the hollow plunger facing the valve body.

The nozzle in the nozzle body is preferably microstructured, i.e. produced by microtechnology. Microstructured nozzle bodies are disclosed for example in WO-94/07607; reference is hereby made to the contents of this specification, particularly FIG. 1 therein and the associated description.

The nozzle body consists for example of two sheets of glass and/or silicon firmly joined together, at least one of which has one or more microstructured channels which connect the nozzle inlet end to the nozzle outlet end. At the nozzle outlet end there is at least one round or non-round opening 2 to 10 microns deep and 5 to 15 microns wide, the depth preferably being 4.5 to 6.5 microns while the length is preferably 7 to 9 microns.

In the case of a plurality of nozzle openings, preferably two, the directions of spraying of the nozzles in the nozzle body may extend parallel to one another or may be inclined relative to one another in the direction of the nozzle opening. In a nozzle body with at least two nozzle openings at the outlet end the directions of spraying may be at an angle of 20 to 160° to one another, preferably 60 to 150°, most preferably 80 to 100°. The nozzle openings are preferably arranged at a spacing of 10 to 200 microns, more preferably at a spacing of 10 to 100 microns, most preferably 30 to 70 microns. Spacings of 50 microns are most preferred. The directions of spraying will therefore meet in the vicinity of the nozzle openings.

The liquid pharmaceutical preparation strikes the nozzle body with an entry pressure of up to 600 bar, preferably 200 to 300 bar, and is atomised into an inhalable aerosol through the nozzle openings. The preferred particle or droplet sizes of the aerosol are up to 20 microns, preferably 3 to 10 microns.

The locking mechanism contains a spring, preferably a cylindrical helical compression spring, as a store for the mechanical energy. The spring acts on the power takeoff flange as an actuating member the movement of which is determined by the position of a locking member. The travel of the power takeoff flange is precisely limited by an upper and lower stop. The spring is preferably biased, via a power step-up gear, e.g. a helical thrust gear, by an external torque which is produced when the upper housing part is rotated counter to the spring housing in the lower housing part. In this case, the upper housing part and the power takeoff flange have a single or multiple V-shaped gear.

The locking member with engaging locking surfaces is arranged in a ring around the power takeoff flange. It consists, for example, of a ring of plastic or metal which is inherently radially elastically deformable. The ring is arranged in a plane at right angles to the atomiser axis. After the biasing of the spring, the locking surfaces of the locking member move into the path of the power takeoff flange and prevent the spring from relaxing. The locking member is actuated by means of a button. The actuating button is connected or coupled to the locking member. In order to actuate the locking mechanism, the actuating button is moved parallel to the annular plane, preferably into the atomiser; this causes the deformable ring to deform in the annual plane. Details of the construction of the locking mechanism are given in WO 97/20590.

The lower housing part is pushed axially over the spring housing and covers the mounting, the drive of the spindle and the storage container for the fluid.

When the atomiser is actuated the upper housing part is rotated relative to the lower housing part, the lower housing part taking the spring housing with it. The spring is thereby compressed and biased by means of the helical thrust gear and the locking mechanism engages automatically. The angle of rotation is preferably a whole-number fraction of 360 degrees, e.g. 180 degrees. At the same time as the spring is biased, the power takeoff part in the upper housing part is moved along by a given distance, the hollow plunger is withdrawn inside the cylinder in the pump housing, as a result of which some of the fluid is sucked out of the storage container and into the high pressure chamber in front of the nozzle.

If desired, a number of exchangeable storage containers which contain the fluid to be atomised may be pushed into the atomiser one after another and used in succession. The storage container contains the aqueous aerosol preparation according to the invention.

The atomising process is initiated by pressing gently on the actuating button. As a result, the locking mechanism opens up the path for the power takeoff member. The biased spring pushes the plunger into the cylinder of the pump housing. The fluid leaves the nozzle of the atomiser in atomised form.

Further details of construction are disclosed in PCT Applications WO 97/12683 and WO 97/20590, to which reference is hereby made.

The components of the atomiser (nebuliser) are made of a material which is suitable for its purpose. The housing of the atomiser and—if its operation permits, other parts as well are preferably made of plastics, e.g. by injection moulding. For medicinal purposes, physiologically safe materials are used.

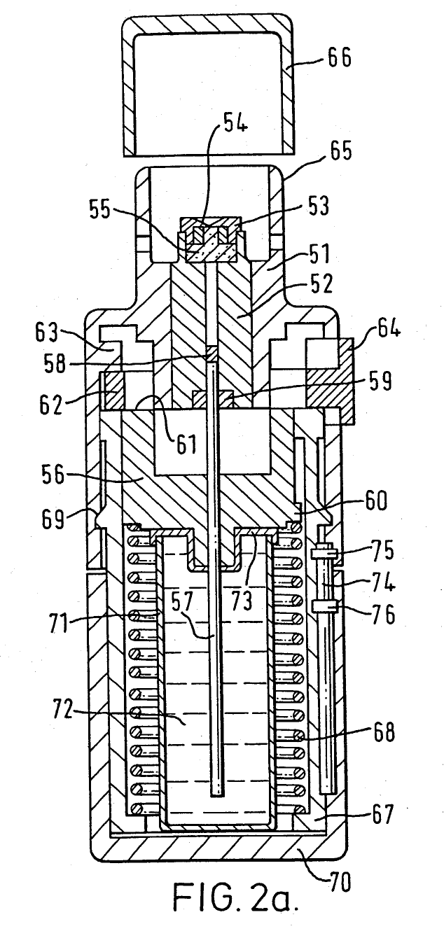

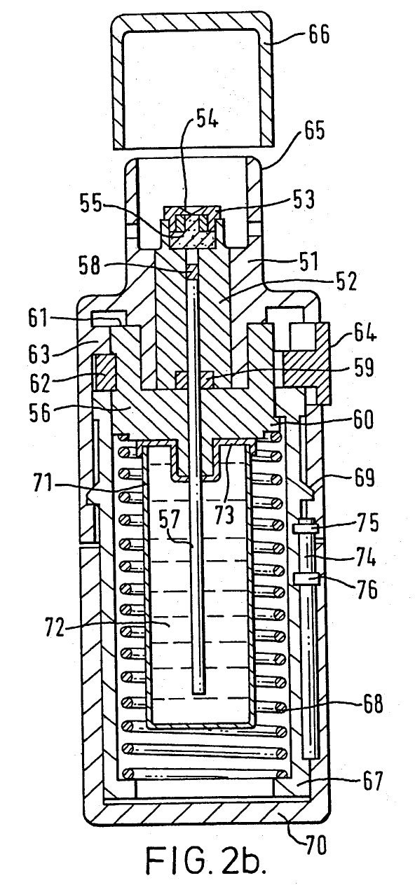

FIG. 2a and FIG. 2b, attached to this patent application, are identical to FIGS. 6a/b of WO 97/12687, and show the nebuliser (Respimat®) which can advantageously be used for inhaling the aqueous aerosol preparations according to the invention.

FIG. 2a shows a longitudinal section through the atomiser with the spring biased while FIG. 2b shows a longitudinal section through the atomiser with the spring relaxed.

The upper housing part (51) contains the pump housing (52) on the end of which is mounted the holder (53) for the atomiser nozzle. In the holder is the nozzle body (54) and a filter (55). The hollow plunger (57) fixed in the power takeoff flange (56) of the locking mechanism projects partially into the cylinder of the pump housing. At its end the hollow plunger carries the valve body (58). The hollow plunger is sealed off by means of the seal (59). Inside the upper housing part is the stop (60) on which the power takeoff flange abuts when the spring is relaxed. On the power takeoff flange is the stop (61) on which the power takeoff flange abuts when the spring is biased. After the biasing of the spring the locking member (62) moves between the stop (61) and a support (63) in the upper housing part. The actuating button (64) is connected to the locking member. The upper housing part ends in the mouthpiece (65) and is sealed off by means of the protective cover (66) which can be placed thereon.

The spring housing (67) with compression spring (68) is rotatably mounted on the upper housing part by means of the snap-in lugs (69) and rotary bearing. The lower housing part (70) is pushed over the spring housing. Inside the spring housing is the exchangeable storage container (71) for the fluid (72) which is to be atomised. The storage container is sealed off by the stopper (73) through which the hollow plunger projects into the storage container and is immersed at its end in the fluid (supply of active substance solution).

The spindle (74) for the mechanical counter is mounted in the covering of the spring housing. At the end of the spindle facing the upper housing part is the drive pinion (75). The slider (76) sits on the spindle.

The nebuliser described above is suitable for nebulising the aerosol preparations according to the invention to produce an aerosol suitable for inhalation.

If the formulation according to the invention is nebulised using the method described above (with the Respimat® device) the quantity delivered should correspond to a defined quantity with a tolerance of not more than 25%, preferably 20% of this amount in at least 97%, preferably at least 98% of all operations of the inhaler (spray actuations). Preferably, between 5 and 30 mg of formulation, most preferably between 5 and 20 mg of formulation are delivered as a defined mass on each actuation.

However, the formulation according to the invention may also be nebulised by means of inhalers other than those described above, e.g. jet stream inhalers.

Accordingly, in a further aspect, the invention relates to pharmaceutical formulations in the form of propellant-free inhalable solutions or suspensions as described above combined with a device suitable for administering these formulations, preferably in conjunction with the Respimat® device. Preferably, the invention relates to propellant-free inhalable solutions or suspensions characterised by the combination of active substances 1 and 2 according to the invention in conjunction with the device known by the name Respimat®. In addition, the present invention relates to the above-mentioned devices for inhalation, preferably the Respimat® device, characterised in that they contain the propellant-free inhalable solutions or suspensions according to the invention as described hereinbefore.

The propellant-free inhalable solutions or suspensions according to the invention may take the form of concentrates or sterile inhalable solutions or suspensions ready for use, as well as the above-mentioned solutions and suspensions designed for use in a Respimat® device. Formulations ready for use may be produced from the concentrates, for example, by the addition of isotonic saline solutions. Sterile formulations ready for use may be administered using energy-operated free-standing or portable nebulisers which produce inhalable aerosols by means of ultrasound or compressed air by the Venturi principle or other principles.

Accordingly, in another aspect, the present invention relates to pharmaceutical compositions in the form of propellant-free inhalable solutions or suspensions as described hereinbefore which take the form of concentrates or sterile formulations ready for use, combined with a device suitable for administering these solutions, characterised in that the device is an energy-operated free-standing or portable nebuliser which produces inhalable aerosols by means of ultrasound or compressed air by the Venturi principle or other methods.

The Examples which follow serve to illustrate the present invention in more detail without restricting the scope of the invention to the following embodiments by way of example.

First, the preparation of compounds 1 used within the scope of the present invention which are not known in the art will be described.

Preparation of the Compounds of Formula 1:

1.a.: 2,2-Diphenypropionic Acid Chloride:

52.08 g (0.33 mol) of oxalyl chloride are slowly added dropwise at 20° C. to a suspension of 25.0 g (0.11 mol) of 2,2-diphenylpropionic acid, 100 ml of dichloromethane and 4 drops of dimethylformamide. The mixture is stirred for 1 h at 20° C. and 0.5 h at 50° C. The solvent is distilled off and the residue remaining is used in the next step without any further purification.

1.b.: Scopine 2,2-diphenylpropionate:

The residue obtained from step 1.a. is dissolved in 100 ml of dichloromethane and at 40° C. a solution of 51.45 g (0.33 mol) of scopine in 200 ml of dichloromethane is added dropwise thereto. The resulting suspension is stirred for 24 h at 40° C., then the precipitate formed is suction filtered and the filtrate is extracted first with water, then with aqueous hydrochloric acid. The combined aqueous phases are made alkaline with aqueous sodium carbonate solution, extracted with dichloromethane, the organic phase is dried over Na₂SO₄, evaporated to dryness and the hydrochloride is precipitated from the residue. The product is purified by recrystallisation from acetonitrile.

Yield: 20.85 g (=47% of theory)

DC: Rf value: 0.24 (eluant: sec. butanol/formic acid/water 75:15:10);

m.p.: 203-204° C.

1.c: Scopine 2,2-diphenylpropionate Methobromide:

11.98 g (0.033 mol) of the compound of step 1.b, 210 ml of acetonitrile, 70 ml of dichloromethane and 20.16 g (0.1 mol) of 46.92% bromomethane in acetonitrile are combined at 20° C. and left to stand for 3 days. The solution is evaporated to dryness and the residue is recrystallised from isopropanol.

Yield: 11.34 g (=75% of theory); m.p.: 208-209° C. C₂₄H₂₈NO₃xBr (458.4);

| | | | | |
|---|---|---|---|---|
| Elemental analysis: | calculated: | C (62.89) | H (6.16) | N (3.06) |
| | found: | C (62.85) | H (6.12) | N (3.07). |

The salts 1 wherein X⁻ denotes an anion with a single negative charge other than bromide may be obtained in a manner similar to step 1.3.

### EXAMPLES OF FORMULATIONS

A) Inhalable Powder

| | Ingredients | μg per capsule |
|---|---|---|
| 1) | 1′-bromide | 200 |
| | compound 2 | 200 |
| | lactose | 4600 |
| | Total | 5000 |
| 2) | 1′-bromide | 100 |
| | compound 2 | 125 |
| | lactose | 4775 |
| | Total | 5000 |
| 3) | 1′-bromide | 50 |
| | compound 2 | 250 |
| | lactose | 4700 |
| | Total | 5000 |
| 4) | 1′-bromide | 130 |
| | compound 2 | 200 |
| | lactose | 4670 |
| | Total | 5000 |

B) Propellant-Driven Inhalable Aerosols:

| | Ingredients | % by weight |
|---|---|---|
| 1) | 1′-bromide | 0.020 |
| | compound 2 | 0.066 |
| | soya lecithin | 0.2 |
| | TG 134a:TG 227 = 2:3 | ad 100 |
| 2) | 1′-bromide | 0.039 |
| | compound 2 | 0.033 |
| | absolute ethanol | 0.5 |
| | isopropyl myristate | 0.1 |
| | TG 227 | ad 100 |

What is claimed is:

1) A pharmaceutical composition of matter comprising, as an active substance one or more, salt of formula 1 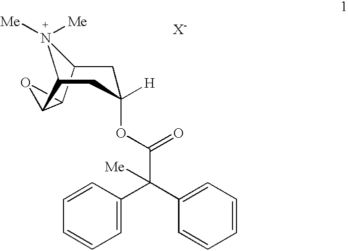
wherein
X⁻ denotes an anion with a single negative charge,
in combination with, also as active substance, one or more compound of formula (2) 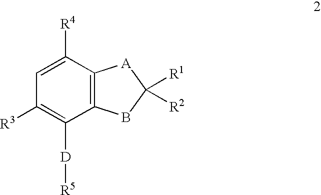
wherein
R¹ denotes hydrogen, methyl, ethyl, n-butyl, i-butyl, phenyl, 2-ethylphenyl, 2-i-propylphenyl, benzyl, 4-pyridyl, 2-pyridyl, —CO-phenyl, CN, or
together with R² denotes a butylene or pentylene bridge;
R² denotes hydrogen, methyl, ethyl, or
together with R¹ denotes a butylene or pentylene bridge, or
together with R¹³ denotes a single bond or a butylene bridge;
R³ denotes hydrogen;
R⁴ denotes methoxy;
R⁵ denotes cyclohexyl, phenyl, 3-methoxycarbonylphenyl, 4-methoxycarbonylphenyl, 3-carboxyphenyl, 4-carboxyphenyl, CN, —COOH, —COOMe, —COOEt, 3,5-dichloro-pyridin-4-yl, 4-pyridyl or 4-pyridyl-N-oxide;
A denotes oxygen or —CH₂—;
B denotes oxygen or one of the groups —C(R¹²)(R¹³) or —CH(R¹⁵)—CH(R¹⁷);
D denotes a group selected from —CH₂—CH₂—, —CH(Ph)—CH₂—, —CONH—, —CO—CH₂—, —CH═CH—, —C(Ph)═CH—, —C(CR¹⁸)(CR¹⁹)—X—, —C(R^{19a})═Y—, —C═C— or phenylene;
R¹² denotes hydrogen, methyl, ethyl, i-propyl, phenyl or —CH₂—COR^{x};
R¹³ denotes hydrogen or
together with R² denotes a single bond or a butylene bridge
R¹⁵ denotes hydrogen or
together with R¹⁷ denotes a single bond;
R¹⁷ denotes hydrogen or
together with R¹⁵ denotes a single bond;
R¹⁸ denotes hydrogen or methyl;
R¹⁹ denotes hydrogen, methoxy, phenyl or CN;
R^{19a} denotes hydrogen, methyl or phenyl;
R^{x} denotes hydroxy, ethoxy, benzyloxy, 2-phenylethyloxy, 4-methylpiperazin-1-yl, 4-phenylpiperazin-1-yl, N-tetrahydroisoquinolinyl, —NH-phenyl, —NH-benzyl, —NH—CH₂-(4-methoxyphenyl), —NH—CH₂-(4-fluorophenyl), —NH—CH₂-(4-chlorophenyl), —NH—CH₂-(2-chlorophenyl), —NH-(3-pyridyl), —NH—CH₂-(2-pyridyl), —NH—CH₂-(3-pyridyl), —NH—CH₂-(4-pyridyl), —NH-(3,5-dichloropyridin-4-yl) or —NH-(2-pyrimidinyl);
X denotes —CH₂—, —S— or —NH—
Y denotes CH, CCN, CCOOEt or CHCONH,
optionally in the form of the individual optical isomers, mixtures thereof or racemates and optionally in the form of the pharmacologically acceptable acid addition salts thereof, optionally in the form of the solvates or hydrates thereof, and a pharmaceutically acceptable carrier or excipient.

2) The pharmaceutical composition of matter as recited in claim 1, characterised in that the compound of formula 2 is a compound of formula 2a, 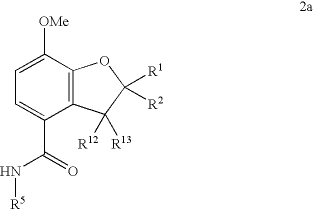
wherein
R¹ denotes hydrogen, n-butyl, benzyl, 4-pyridyl, 2-pyridyl, —CO-phenyl or CN;
R² denotes hydrogen or together with R¹³ denotes a single bond;
R⁵ denotes cyclohexyl, phenyl, 3,5-dichloro-pyridin-4-yl or 4-pyridyl;
R¹² denotes hydrogen, methyl, ethyl, i-propyl, phenyl or —CH₂—COR^{x};
R¹³ denotes hydrogen or together with R² denotes a single bond;
R^{x} denotes hydroxy, ethoxy, benzyloxy, 2-phenylethoxy, 4-methylpiperazin-1-yl, 4-phenylpiperazin-1-yl, N-tetrahydroisoquinolinyl, —NH-phenyl, —NH-benzyl, —NH—CH₂-(4-methoxyphenyl), —N H—CH₂-(4-fluorophenyl), —N H—CH₂-(4-chlorophenyl), —NH—CH₂-(2-chlorophenyl), —NH-(3-pyridyl), —NH—CH₂-(2-pyridyl), —NH—CH₂-(3-pyridyl), —NH—CH₂-(4-pyridyl), —NH-(3,5-dichloropyridin-4-yl) or —NH-(2-pyrimidinyl),
optionally in the form of the individual optical isomers, mixtures thereof or racemates and optionally in the form of the pharmacologically acceptable acid addition salts thereof.

3) The pharmaceutical composition of matter as recited in claim 1, characterised in that the compound of formula 2 is a compound of formula 2b, 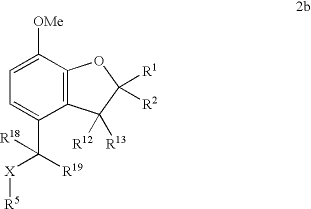
wherein
R¹ denotes hydrogen, methyl, ethyl or 4-pyridyl, or
together with R² denotes a butylene bridge;
R² denotes hydrogen, methyl, ethyl, or
together with R¹ denotes a butylene bridge, or
together with R¹³ denotes a single bond;
R⁵ denotes 3,5-dichloro-pyridin-4-yl or 4-pyridyl;
R¹² denotes hydrogen or methyl;
R¹³ denotes hydrogen or
together with R² denotes a single bond;
R¹⁸ denotes hydrogen or methyl;
R¹⁹ denotes hydrogen, methoxy, phenyl or CN;
X denotes —CH₂—, —S— or —NH—,
optionally in the form of the individual optical isomers, mixtures thereof or racemates and optionally in the form of the pharmacologically acceptable acid addition salts thereof.

4) The pharmaceutical composition of matter as recited in claim 1, characterised in that the compound of formula 2 is a compound of formula 2c, 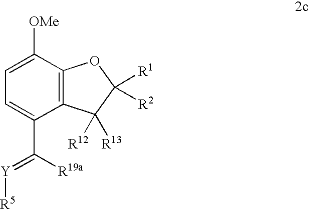
wherein
R¹ denotes hydrogen, methyl, ethyl, phenyl, 4-pyridyl, 2-pyridyl, or
together with R² denotes a butylene or pentylene bridge;
R² denotes hydrogen, methyl, ethyl, or
together with R¹ denotes a butylene or pentylene bridge, or
together with R¹³ denotes a single bond;
R⁵ denotes 3-methoxycarbonylphenyl, 4-methoxycarbonylphenyl, 3-carboxyphenyl, 4-carboxyphenyl, CN, —COOEt, 3,5-dichloro-pyridin-4-yl or 4-pyridyl;
R¹² denotes hydrogen or methyl;
R¹³ denotes hydrogen or
together with R² denotes a single bond;
R^{19a} denotes hydrogen, methyl or phenyl;
Y denotes CH, CCN, CCOOEt or CHCONH,
optionally in the form of the individual optical isomers, mixtures thereof or racemates and optionally in the form of the pharmacologically acceptable acid addition salts thereof.

5) The pharmaceutical composition of matter as recited in claim 1, characterised in that the compound of formula 2 is a compound of formula 2d, 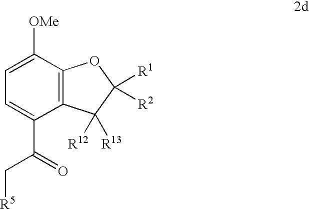
wherein
R¹ denotes hydrogen, methyl, ethyl, n-butyl, i-butyl, phenyl, 2-ethylphenyl, 2-1-propylphenyl, 4-pyridyl, 2-pyridyl, —CO-phenyl, CN, or
together with R denotes a butylene or pentylene bridge;
R² denotes hydrogen, methyl, ethyl, or
together with R¹ denotes a butylene or pentylene bridge, or
together with R¹³ denotes a single bond or a butylene bridge;
R⁵ denotes phenyl, 3,5-dichloro-pyridin-4-yl or 4-pyridyl;
R¹² denotes hydrogen, methyl, phenyl or —CH₂—COR^{x};
R¹³ denotes hydrogen or
together with R² denotes a single bond or a butylene bridge
R^{x} denotes ethoxy,
optionally in the form of the individual optical isomers, mixtures thereof or racemates and optionally in the form of the pharmacologically acceptable acid addition salts thereof.

6) The pharmaceutical composition of matter as recited in claim 1, characterised in that the compound of formula 2 is a compound of formula 2e, 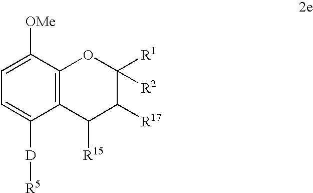
wherein
R¹ denotes methyl or
together with R² denotes a butylene or pentylene bridge;
R² denotes methyl or
together with R¹ denotes a butylene or pentylene bridge;
R⁵ denotes 3,5-dichloro-pyridin-4-yl or 4-pyridyl;
D denotes a group selected from —CONH—, —CO—CH₂— or —CH═CH—;
R¹⁵ denotes hydrogen or
together with R¹⁷ denotes a single bond;
R¹⁷ denotes hydrogen or
together with R¹⁵ denotes a single bond,
optionally in the form of the individual optical isomers, mixtures thereof or racemates and optionally in the form of the pharmacologically acceptable acid addition salts thereof.

7) The pharmaceutical composition of matter as recited in claim 1, characterised in that the compound of formula 2 is a compound of formula 2f, 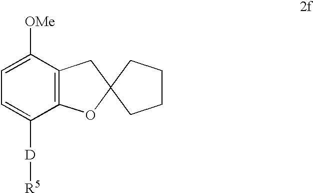
wherein
R⁵ denotes 3,5-dichloro-pyridin-4-yl or 4-pyridyl;
D denotes a group selected from —CONH—, —CO—CH₂— or —CH═CH—,
optionally in the form of the individual optical isomers, mixtures thereof or racemates and optionally in the form of the pharmacologically acceptable acid addition salts thereof.

8) The pharmaceutical composition of matter as recited in claim 1, characterised in that the compound of formula 2 is a compound of formula 2g, 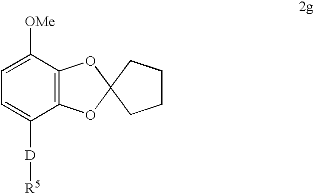
wherein
R⁵ may represent 3,5-dichloro-pyridin-4-yl or 4-pyridyl;
D may represent a group selected from —CH₂—CH₂—, —CH(Ph)—CH₂—, —CONH—, —CO—CH₂—, —CH═CH— or —C(Ph)═CH—,
optionally in the form of the individual optical isomers, mixtures thereof or racemates and optionally in the form of the pharmacologically acceptable acid addition salts thereof.

9) The pharmaceutical composition of matter as recited in claim 1, according to claim 1, characterised in that the compound of formula 2 is a compound of formula 2h, 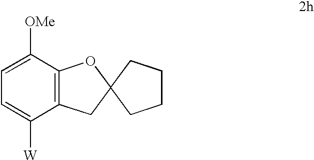
wherein
W denotes a group selected from among 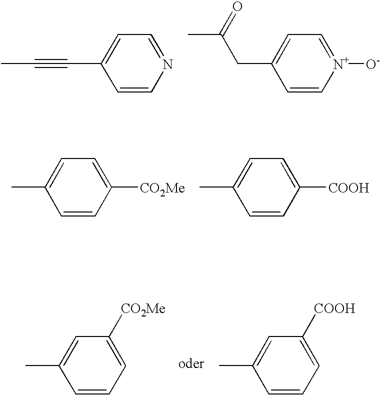
optionally in the form of the individual optical isomers, mixtures thereof or racemates and optionally in the form of the pharmacologically acceptable acid addition salts thereof.

10) The pharmaceutical composition of matter as recited in claim 1, characterised in that the active substances 1 and 2 are present either together in a single formulation in the composition or in two separate formulations in the composition.

11) The pharmaceutical composition of matter as recited in claim 1, characterised in that compound of formula 1 is present in the form of the chloride, bromide, methanesulphonate or para-toluenesulphonate.

12) The pharmaceutical composition of matter as recited in claim 1, characterised in that compound of formula 1 is present in the form of the chloride, bromide or methanesulphonate.

13) The pharmaceutical composition of matter as recited in claim 1, characterised in that the weight ratios of active substances 1 to 2 are in the range from about 1:300 to about 80:1.

14) The pharmaceutical composition of matter as recited in claim 1, characterised in that a single administration corresponds to a dose of the combination of active substances 1 and 2 of about 0.01 to 1000 μg.

15) The pharmaceutical composition of matter as recited in claim 1, characterised in that it is in a form suitable for inhalation.

16) The pharmaceutical composition of matter as recited in claim 15, characterised in that it is an inhalable powder, a propellant-containing metered-dose aerosol or a propellant-free inhalable solution or suspension.

17) The pharmaceutical composition of matter as recited in claim 16, characterised in that it is an inhalable powder with a maximum average particle size of up to about 250 μm.

18) The pharmaceutical composition of matter as recited in claim 16, characterised in that it is a propellant-free inhalable solution which contains water, ethanol or a mixture of water and ethanol as solvent.

19) The pharmaceutical composition of matter as recited in claim 18, characterised in that the pH of the solution is about 2-7.

20) A method for treating inflammatory or obstructive respiratory complaints in a warm blooded animal which comprises administering to said animal a therapeutically effective amount of the pharmaceutical composition of matter as recited in claim 1.
